SEQUENCE LISTING (37 sequences)

SEQ ID NO: 1 - protein, 389 aa, Homo sapiens
MAEPDPSHPLETQAGKVQEAQDSDSDSEGGAAGGEADMDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 2 - protein, 352 aa, Homo sapiens
MDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 3 - protein, 389 aa, Mus musculus
MAEPDPSDPLETQAGKVQEAQDSDSDTEGGATGGEAEMDFLRNLFTQTLGLGSQKERLLDELTLEGVTRYMQSERCRKVICLVGAGISTSAGIPDFRSPSTGLYANLEKYHLPYPEAIFEISYFKKHPEPFFALAKELYPGQFKPTICHYFIRLLKEKGLLLRCYTQNIDTLERVAGLEPQDLVEAHGTFYTSHCVNTSCRKEYTMGWMKEKIFSEATPRCEQCQSVVKPDIVFFGENLPPRFFSCMQSDFSKVDLLIIMGTSLQVQPFASLISKAPLATPRLLINKEKTGQTDPFLGMMMGLGGGMDFDSKKAYRDVAWLGDCDQGCLALADLLGWKKELEDLVRREHANIDAQSGSQAPNPSTTISPGKSPPPAKEAARTKEKEEQQ

SEQ ID NO: 4 - protein, 357 aa, Saccharomyces cerevisiae
MSVSTASTEMSVRKIAAHMKSNPNAKVIFMVGAGISTSCGIPDFRSPGTGLYHNLARLKLPYPEAVFDVDFFQSDPLPFYTLAKELYPGNFRPSKFHYLLKLFQDKDVLKRVYTQNIDTLERQAGVKDDLIIEAHGSFAHCHCIGCGKVYPPQVFKSKLAEHPIKDFVKCDVCGELVKPAIVFFGEDLPDSFSETWLNDSEWLREKITTSGKHPQQPLVIVVGTSLAVYPFASLPEEIPRKVKRVLCNLETVGDFKANKRPTDLIVHQYSDEFAEQLVEELGWQEDFEKILTAQGGMGDNSKEQLLEIVHDLENLSLDQSEHESADKKDKKLQRLNGHDSDEDGASNSSSSQKAAKE

SEQ ID NO: 5 - protein, 355 aa, Drosophila melanogaster
MDKVRRFFANTLHLGGSSDAKEEVKVEKVIPDLSFDGFAEHWRVHGFRKIVTMVGAGISTSAGIPDFRSPGSGLYSNLKKYELPHPTAIFDLDYFEKNPAPFFALAKELYPGSFIPTPAHYFIRLLNDKGLLQRHYTQNIDTLDRLTGLPEDKIIEAHGSFHTNHCIKCRKEYDMDWMKAEIFADRLPKCQKCQGVVKPDIVFFGENLPKRFYSSPEEDFQDCDLLIIMGTSLEVQPFASLVWRPGPRCIRLLINRDAVGQASCVLFMDPNTRSLLFDKPNNTRDVAFLGDCDAGVMALAKALGWDQELQQLITSERKKLSGSQNSEELQQGKEKPQSDPDKMTSGDRDKKDASL

SEQ ID NO: 6 - protein, 655 aa, Homo sapiens
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 7 - protein, 652 aa, Mus Musculus
MAEAPQVVETDPDFEPLPRQRSCTWPLPRPEFNQSNSTTSSPAPSGGAAANPDAAASLASASAVSTDFMSNLSLLEESEDFARAPGCVAVAAAAAASRGLCGDFQGPEAGCVHPAPPQPPPTGPLSQPPPVPPSAAAAAGPLAGQPRKTSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRGRAAKKKASLQSGQEGPGDSPGSQFSKWPASPGSHSNDDFDNWSTFRPRTSSNASTISGRLSPIMTEQDDLGDGDVHSLVYPPSAAKMASTLPSLSEISNPENMENLLDNLNLLSSPTSLTVSTQSSPGSMMQQTPCYSFAPPNTSLNSPSPNYSKYTYGQSSMSPLPQMPMQTLQDSKSSYGGLNQYNCAPGLLKELLTSDSPPHNDIMSPVDPGVAQPNSRVLGQNVMMGPNSVMPAYGSQASHNKMMNPSSHTHPGHAQQTASVNGRTLPHVVNTMPHTSAMNRLTPVKTPLQVPLSHPMQMSALGSYSSVSSCNGYGRMGVLHQEKLPSDLDGMFIERLDCDMESIIRNDLMDGDTLDFNFDNVLPNQSFPHSVKTTTHSWVSG

SEQ ID NO: 8 - DNA, 64 bp, Artificial sequence
  Primer
gatccccgaaggagtgacacgctacattcaagagatgtagcgtgtcactccttctttttggaaa

SEQ ID NO: 9 - DNA, 64 bp, Artificial Sequence
  Primer
agcttttccaaaaagaaggagtgacacgctacatctcttgaatgtagcgtgtcactccttcggg

SEQ ID NO: 10 - DNA, 20 bp, Artificial Sequence
  Primer
tgattctgctgcccttctgt

SEQ ID NO: 11 - DNA, 21 bp, Artificial Sequence
  Primer
caagaacagcaacgagtaccg

SEQ ID NO: 12 - DNA, 19 bp, Artificial Sequence
  Primer
gcttttgtcacatgcaggt

SEQ ID NO: 13 - DNA, 19 bp, Artificial Sequence
  Primer
gatgcctttgtgggaacct

SEQ ID NO: 14 - DNA, 23 bp, Artificial Sequence
  Primer
acccttcaccaatgactcctatg

SEQ ID NO: 15 - DNA, 21 bp, Artificial Sequence
  Primer
ggctctatggattacccaagc

SEQ ID NO: 16 - DNA, 20 bp, Artificial Sequence
  Primer
tcagctctgtggacctctcc

SEQ ID NO: 17 - DNA, 19 bp, Artificial Sequence
  Primer
agaaccaccaaagcggaaa

SEQ ID NO: 18 - DNA, 20 bp, Artificial Sequence
  Primer
agccaaccatctgccactac

SEQ ID NO: 19 - DNA, 20 bp, Artificial Sequence
  Primer
tgctactcatcttgggacct

SEQ ID NO: 20 - DNA, 20 bp, Artificial Sequence
  Primer
gagcaactgggagagactgg

SEQ ID NO: 21 - DNA, 19 bp, Artificial Sequence
  Primer
cgctggaggttactggaga

SEQ ID NO: 22 - DNA, 19 bp, Artificial Sequence
  Primer
catgggcttcctcagcttc

SEQ ID NO: 23 - DNA, 18 bp, Artificial Sequence
  Primer
agcctaccctcacccaca

SEQ ID NO: 24 - DNA, 20 bp, Artificial Sequence
  Primer
ggacattggacgctctctct

SEQ ID NO: 25 - DNA, 21 bp, Artificial Sequence
  Primer
gtcactggtcaactccagcac

SEQ ID NO: 26 - DNA, 20 bp, Artificial Sequence
  Primer
cgcacagagcactccataaa

SEQ ID NO: 27 - DNA, 19 bp, Artificial Sequence
  Primer
ctgtcgtctgcggtgattt

SEQ ID NO: 28 - DNA, 21 bp, Artificial Sequence
  Primer
tgactgcagcaaatcgcttgg

SEQ ID NO: 29 - DNA, 19 bp, Artificial Sequence
  Primer
ccagtgttcgttcctcgga

SEQ ID NO: 30 - DNA, 20 bp, Artificial Sequence
  Primer
acccttgcatccttcacaag

SEQ ID NO: 31 - DNA, 20 bp, Artificial Sequence
  Primer
tcccacaggagacagaaacc

SEQ ID NO: 32 - DNA, 20 bp, Artificial Sequence
  Primer
ccagcccatcgtgtattctt

SEQ ID NO: 33 - DNA, 18 bp, Artificial Sequence
  Primer
caccagcctttccacacc

SEQ ID NO: 34 - DNA, 18 bp, Artificial Sequence
  Primer
acagcacgggacctgaaa

SEQ ID NO: 35 - DNA, 20 bp, Artificial Sequence
  Primer
cgtcaatgttctgggtgatg

SEQ ID NO: 36 - DNA, 19 bp, Artificial Sequence
  Primer
aacgagtcctcccagtcca

SEQ ID NO: 37 - DNA, 20 bp, Artificial Sequence
  Primer
cgctcagtcacatcaaacac

(19) United States
(12) Patent Application Publication
(10) Pub. No.: US 2009/0142335 A1
(43) Pub. Date: Jun. 4, 2009

(54) Methods of diagnosis and treatment of metabolic disorders

(75) Inventors: C. Ronald Kahn, West Newton, MA (US); Enxuan Jing, Chestnut Hill, MA (US); Stephane Gesta, Boston, MA (US)

Correspondence Address: CLARK & ELBING LLP, 101 FEDERAL STREET, BOSTON, MA, 02110, US

(73) Assignee: Joslin Diabetes Center, Boston, MA (US)

(21) Appl. No.: 12/220,714

(22) Filed: Jul. 28, 2008

Related U.S. Application Data
(63) Continuation-in-part of application No. 11/883,867.
(60) Provisional application No. 60/962,275, filed on Jul. 27, 2007.
(60) Provisional application No. 60/687,215, filed on Jun. 3, 2005.
(60) Provisional application No. 60/652,934, filed on Feb. 15, 2005.

(51) Int. Cl. A61K 39/395; C12Q 1/34; C12Q 1/68; G01N 33/53; A61P 3/00; A61K 38/46; A61K 31/7052; A61K 31/05

(52) U.S. Cl. 424/130.1; 435/18; 435/6; 435/7.1; 424/94.6; 514/44; 514/733

(57) ABSTRACT

The invention features diagnostic methods for metabolic disorders (e.g., diabetes and obesity), methods for screening for compounds useful in the treatment of metabolic disorders, and methods for treatment of metabolic disorders that involve sirtuin2.

### CROSS-REFERENCE TO RELATED APPLICATIONS

This application claims benefit of U.S. Provisional Application No. 60/962,275, filed Jul. 27, 2007, and is also a continuation-in-part of U.S. application Ser. No. 11/883,867, which is the national stage of PCT/US2006/005493, filed Feb. 15, 2006, which, in turn, claims benefit of U.S. Provisional Application Nos. 60/687,215, filed Jun. 3, 2005, and 60/652,934, filed Feb. 15, 2005. Each of these applications is hereby incorporated by reference.

### STATEMENT AS TO FEDERALLY SPONSORED RESEARCH

The present research was supported by a grant from the National Institutes of Health (Numbers DK36836-15, DK33201, and DK45935). The U.S. Government may therefore have certain rights to this invention.

### BACKGROUND OF THE INVENTION

The invention relates to the field of metabolic disorders, methods of diagnosing and treating such disorders, and screening methods for identification of compounds useful in treating metabolic disorders.

Metabolic disorders such as obesity are serious health problems. 34% of U.S. adults age 20 and over are considered obese. The prevalence of obesity has increased markedly over the last 30 years. Obesity is a risk factor for developing cardiovascular disease, type II diabetes, cancers including esophageal and colon cancers, asthma, and sleep disorders.

Diabetes mellitus, which results from a loss of insulin action on peripheral tissues, is a metabolic disorder accompanied by alterations in cellular physiology, metabolism, and gene expression and is one of the most common causes of morbidity and mortality in westernized countries (Skyler and Oddo, (2002) Diabetes Metab. Res. Rev. 18 Suppl 3, S21-S26). Although diabetes may arise secondarily to any condition that causes extensive damage to the pancreas (e.g., pancreatitis, tumors, administration of certain drugs such as corticosteroids or pentamidine, iron overload (e.g., hemochromatosis), acquired or genetic endocrinopathies, and surgical excision), the most common forms of diabetes typically arise from primary disorders of the insulin signaling system. There are two major types of diabetes, namely type 1 diabetes (also known as insulin dependent diabetes (IDDM)) and type 2 diabetes (also known as insulin independent or non-insulin dependent diabetes (NIDDM)), which share common long-term complications in spite of their different pathogenic mechanisms.

Given that the strategies currently available for the management of metabolic disorders such as obesity and diabetes are suboptimal, there is a compelling need for treatments that are more effective and are not associated with debilitating side effects.

### SUMMARY OF THE INVENTION

The present invention provides methods that relate to applicants' newly discovered role of sirtuin2 in metabolic disorders. In a first aspect, the invention provides a method of diagnosing a metabolic disorder (e.g., obesity), or a propensity thereto, in a subject (e.g., a human). The method includes analyzing the level of sirtuin2 expression or activity in a sample isolated from the subject, where a decreased level of sirtuin2 expression or activity in the sample relative to the level in a control sample indicates that the subject has the metabolic disorder, or a propensity thereto. The analyzing may include measuring in the sample the amount of sirtuin2 RNA or protein, the histone deacetylase activity of sirtuin2, the deacetylation of Foxo1 by sirtuin2, or the binding of sirtuin2 to Foxo1.

In another aspect, the invention provides a method of identifying a candidate compound useful for treating a metabolic disorder (e.g., obesity) in a subject. The method includes contacting a sirtuin2 protein (e.g., human sirtuin2 protein) with a compound (e.g., a compound selected from a chemical library); and measuring the activity of the sirtuin2 (e.g., binding to or deacetylation of Foxo1), where an increase in sirtuin2 activity in the presence of the compound relative to the sirtuin2 activity in the absence of the compound identifies the compound as a candidate compound for treating a metabolic disorder in a subject. The method may be performed in vivo (for example, in a cell or animal) or in vitro.

In another aspect, the invention provides a method of identifying a candidate compound useful for treating a metabolic disorder (e.g., obesity) in a subject. The method includes contacting a sirtuin2 protein (e.g., human sirtuin2 protein) with a compound (e.g., a compound selected from a chemical library); and measuring the binding of the compound to sirtuin2, where specific binding of the compound to the sirtuin2 protein identifies the compound as a candidate compound for treating a metabolic disorder in a subject.

In a related aspect, the invention provides a method for identifying a candidate compound useful for treating a metabolic disorder (e.g., obesity) in a subject. The method includes contacting a cell or cell extract including a polynucleotide encoding sirtuin2 (e.g., human sirtuin2) with a compound (e.g., a compound selected from a chemical library); and measuring the level of sirtuin2 expression in the cell or cell extract, where an increased level of sirtuin2 expression in the presence of the compound relative to the level in the absence of the compound identifies the compound as a candidate compound for treating a metabolic disorder in a subject.

In another aspect, the invention provides a method of treating a metabolic disorder (e.g., obesity) in a subject (e.g., a human). The method includes administering to the subject a composition that increases sirtuin2 expression or activity, for example, sirtuin2, or an active fragment thereof, a polynucleotide encoding sirtuin2 or an active fragment thereof, a sirtuin2-activating compound such as resveratrol or a derivative thereof, or a compound identified using the methods described herein. The increased sirtuin2 activity includes binding to or deacetylation of Foxo1. In some embodiments, the nucleic acid coding for the sirtuin2 protein is capable of expressing sirtuin2 in a desired tissue (e.g., adipose tissue).

In another aspect, the invention provides a kit for treating a subject with a metabolic disorder. The kit includes a composition that increases sirtuin2 expression or activity (e.g., binding to or deacetylation of Foxo1); and instructions for administering the composition to a subject with a metabolic disorder.

By “sirtuin2” is meant a polypeptide with at least 30%, 40%, 50%, 60%, 70%, 80%, 90%, 95%, or 99% sequence identity to SEQ ID NO:1, SEQ ID NO:2, or a fragment thereof (FIG. 13) or a polypeptide encoded by a polynucleotide that hybridizes to a polynucleotide encoding SEQ ID NO:1, SEQ ID NO:2, or a fragment thereof.

By “Foxo1” is meant a polypeptide with at least 30%, 40%, 50%, 60%, 70%, 80%, 90%, 95%, or 99% sequence identity to SEQ ID NO:6, or a fragment thereof, or a polypeptide encoded by a polynucleotide that hybridizes to a polynucleotide encoding SEQ ID NO:6, or a fragment thereof (FIG. 14).

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “hybridize” is meant pair to form a double-stranded complex containing complementary paired nucleic acid sequences, or portions thereof, under various conditions of stringency. (See, e.g., Wahl and Berger, (1987) Methods Enzymol. 152, 399-407; Kimmel, (1987) Methods Enzymol. 152, 507-511). For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 1.5 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180 (1977)); Grunstein and Hogness ((1975) Proc. Natl. Acad. Sci. USA 72, 3961); Ausubel et al. (Current Protocols in Molecular Biology, Wiley Interscience, New York (2001)); Berger and Kimmel (Guide to Molecular Cloning Techniques, Academic Press, New York, (1987)); and Sambrook et al. (Molecular Cloning: A Laboratory Manual, Cold Spring Harbor Laboratory Press, New York). Preferably, hybridization occurs under physiological conditions. Typically, complementary nucleobases hybridize via hydrogen bonding, which may be Watson-Crick, Hoogsteen, or reversed Hoogsteen hydrogen bonding, between complementary nucleobases. For example, adenine and thymine are complementary nucleobases that pair through the formation of hydrogen bonds.

By “fragment” is meant a chain of at least 4, 5, 6, 8, 10, 15, 20, or 25 amino acids or nucleotides which comprises any portion of a larger protein or polynucleotide.

By “biological sample” or “sample” is meant a sample obtained from an organism or from components (e.g., cells) of an organism. The sample may be of any biological tissue or fluid. Frequently the sample will be a “clinical sample” which is a sample derived from a subject. Such samples include, but are not limited to, sputum, blood, blood cells (e.g., white cells), tissue or fine needle biopsy samples, urine, peritoneal fluid, and pleural fluid, or cells. Biological samples may also include sections of tissues such as frozen sections taken for histological purposes.

By “subject” is meant either a human or non-human animal (e.g., a mammal).

“Treating” a disease or condition in a subject or “treating” a subject having a disease or condition refers to subjecting the individual to a pharmaceutical treatment, e.g., the administration of a drug, such that at least one symptom of the disease or condition is decreased or stabilized.

By “preventing” a disease or condition in a subject is meant reducing or eliminating the risk of developing the disease or condition prior to the appearance of the disease.

By “specifically binds” or “specific binding” is meant a compound or antibody which recognizes and binds a polypeptide of the invention but which does not substantially recognize and bind other molecules in a sample, for example, a biological sample, which naturally includes a polypeptide of the invention.

By “decrease in the level of expression or activity” of a gene is meant a reduction in protein or nucleic acid level or activity in a cell, a cell extract, or a cell supernatant. For example, such a decrease may be due to reduced RNA stability, transcription, or translation, increased protein degradation, or RNA interference. Preferably, this decrease is at least 5%, 10%, 25%, 50%, 75%, 80%, or even 90% of the level of expression or activity under control conditions.

By “increase in the expression or activity” of a gene or protein is meant a positive change in protein or nucleic acid level or activity in a cell, a cell extract, or a cell supernatant. For example, such a increase may be due to increased RNA stability, transcription, or translation, or decreased protein degradation. Preferably, this increase is at least 5%, 10%, 25%, 50%, 75%, 80%, 100%, 200%, or even 500% or more over the level of expression or activity under control conditions.

By a “compound,” “candidate compound,” or “factor” is meant a chemical, be it naturally-occurring or artificially-derived. Compounds may include, for example, peptides, polypeptides, synthetic organic molecules, naturally-occurring organic molecules, nucleic acid molecules, and components or combinations thereof.

By a “metabolic disorder” is meant any pathological condition resulting from an alteration in a mammal's metabolism. Such disorders include those resulting from an alteration in glucose homeostasis resulting, for example, in hyperglycemia. According to this invention, an alteration in glucose level is typically a glucose level that is increased by at least 5%, 10%, 20%, 30%, 40%, 50%, 75%, 100%, 125%, 150%, 200%, or even 250% relative to such levels in a healthy individual under identical conditions. Metabolic disorders include obesity (e.g., Body Mass Index (BMI) greater than 25.0 or 30.0), diabetes (e.g., diabetes type I, diabetes type II, MODY diabetes, and gestational diabetes), and metabolic syndrome.

Other features and advantages of the invention will be apparent from the following Detailed Description, the drawings, and the claims.

### BRIEF DESCRIPTION OF THE DRAWINGS

FIG. 1A is a depiction of acetylation and phosphorylation sites of mouse Foxo1 (SEQ ID NO:7), a transcription factor regulated by its acetylation state.

FIG. 1B is a schematic diagram showing that CBP (cAMP-response element-binding protein-binding protein) regulates Foxo1 activity by acetylating Foxo1, and that PKB (protein kinase B; Akt) phosphorylates the acetylated Foxo1.

FIGS. 2A-2D are a set of graphs and photographs showing expression of sirt2 and stable sirt2 knockdown in 3T3-L1 preadipocytes. FIG. 2A shows Affymetrix microarray analysis performed using mRNA isolated from epididymal adipocytes as described previously (Gesta et al., Proc Natl Acad Sci USA 103, 6676-81 (2006)). To confirm these finding, quantitative real-time PCR was performed as described below.

FIG. 2B shows changes in expression of the different Sirt mRNA during 3T3-L1 white adipocyte differentiation using real-time PCR. sirt2 mRNA was the most abundant in adipocytes, and both Sirt1 and sirt2 had similar pattern of diminishing expression during adipocyte differentiation. FIG. 2C shows shRNA overexpression constructs generated with pSuper-Retro vector. Two shRNA constructs (S-1 and S-2) were tested by targeting different exons of sirt2 genomic sequence. An shGFP RNAi sequence was used as a control. After retroviral infection and selection, 3T3-L1 preadipocytes carrying either shGFP or shsirt2 overexpression constructs were grown to confluence then RNA was extracted to synthesize cDNA and real-time PCR was performed for Sirt 1-3. FIG. 2D shows transient transfection experiments were done using two different shRNA and control shRNA constructs along with either control pBabe or SIRT2-FLAG overexpression. Both shsirt2 (S1 and S2) effectively knockdown the overexpression of SIRT2-FLAG protein. Endogenous sirt2 knockdown was also detected by western blot. All the error bars in this figure refer to Standard Error (SE) of the mean.

FIG. 3 is a graph showing that Sirt2 mRNA is the most abundant isoform among seven family members in 3T3-L1 cells. Real-time PCR was performed using different primers targeting 7 sirt isoforms. Results showed that Sirt2 mRNA is most abundant transcript among 7 family members in 3T3-L1 cells. Among other Sirt transcripts, Sirt6 and Sirt7 have relatively higher mRNA expression level, while Sirt5 mRNA abundance is the lowest among 7 family members.

FIGS. 4A-4C are a set of graphs and images showing that Sirt2 knockdown promotes 3T3-L1 adipocyte differentiation. FIG. 4A shows stable shRNA transfected 3T3-L1 preadipocytes were subjected to differentiation using the standard protocol. Oil Red O staining of shGFP and shsirt2 cells on day 4 of differentiation indicated that shsirt2 had accelerated differentiation with enhanced lipid staining. FIG. 4B shows that, during 3T3-L1 adipocyte differentiation, shsirt2 cells (empty circles) had consistently lower endogenous sirt2 mRNA expression compared with shGFP cells (solid circles). The mRNA expression for various differentiation markers was also determined by real-time PCR. FIG. 4C shows protein expression of different adipocyte differentiation markers determined by Western blotting. Error bars in this figure refer to Standard Error (SE) of the mean.

FIGS. 5A and 5B are photographs showing that Sirt2 overexpression inhibits 3T3-L1 adipocyte differentiation without affecting insulin signaling in preadipocytes.

FIG. 5A shows that, following the differentiation protocol described below, exogenous sirt2 overexpression inhibited adipocyte differentiation as compared with control cells, as shown by Oil Red O staining of stably transfected 3T3-L1 cells with either control pBabe vector or SIRT2-FLAG-pBabe overexpression construct. FIG. 5B shows insulin signaling assessed by western blotting of phospho-Akt, phospho-p38 and phospho-MAP kinase in confluent 3T3-L1 preadipocytes. Stimulation was performed using 10 nM and 100 nM insulin for 5 min.

FIG. 6 is a photomicrograph showing subcellular distribution of Sirt2-FLAG overexpression in 3T3-L1 preadipocytes. Immunocytochemistry was done with an anti-FLAG-FITC antibody. Exogenous Sirt2 overexpression showed similar distribution pattern to previous reports regarding endogenous Sirt2 subcellular localization, which is mainly in the cytoplasm. The lighter signal in the nucleus with a very localized pattern suggests that Sirt2 may trafficking to the nucleus.

FIG. 7 is a set of images showing Sirt2 knockdown promoting FOXO1 acetylation. Non-denaturing total protein extracts from either shGFP or shsirt2 cells were immunoprecipitated with anti-acetylated-Lys antibody and precipitated lysates were blotted with anti-FOXO1 antibody. Total lysate input was detected by western blotting.

FIG. 8 is photograph of a western blot showing that endogenous Sirt2 knockdown induces hyperacetylation of exogenous FoxO1. Cell lines overexpressing FoxO1-FLAG was also stably transfected with either shGFP or shSirt2 overexpression constructs. Lysates from these two cell lines were subjected to immunoprecipitation using anti-FLAG-agarose, to precipitate the he exogenous FoxO1. The precipitated lysate was then applied to an SDS gel. Western blotting using either anti-acetyl-lysine or FoxO1 antibody was then performed. FoxO1 was equally precipitated from both cell lines. Cells carrying shSirt2 had higher acetyl-lysine reactivity, whereas the signal could not be detected in cells with shGFP. Knocking down endogenous Sirt2 thus leads to hyperacetylation of exogenous FoxO1, suggesting that FOXO1 is a substrate of Sirt2.

FIGS. 9A-9D are images showing that SIRT2 interacts with FOXO1 in vitro and sirt2 knockdown promotes FOXO1 phosphorylation and cytosolic localization. FIG. 9A shows non-denaturing lysates from either pBabe control or SIRT2-FLAG overexpression cell lines immunoprecipitated with anti-FLAG-agarose. The precipitated lysates were blotted with anti-FOXO1 antibody. Markedly more FOXO1 protein was precipitated with anti-FLAG-agarose from SIRT2-FLAG overexpressing cells. FIG. 9B shows non-denaturing lysates from HEK293 cells transiently transfected with SIRT2-HA and/or FOXO1-FLAG overexpressing constructs subjected to immunoprecipitation with anti-HA agarose. Western blot of protein eluted from HA-Agarose shows that there is interaction between SIRT2 and FOXO1 in vitro. FIG. 9C shows shGFP or shsirt2 cells acutely (5 or 15 minutes) stimulated with different concentrations of insulin (10 nM and 100 nM) after serum deprivation. Insulin stimulated Akt and GSK3β phosphorylation (5 min stimulation) and FOXO1 phosphorylation (15 min stimulation) were assessed by western blotting. FIG. 9D shows lysates from both shGFP and shsirt2 cells subjected to western blot analysis with anti-FOXO1 antibody, using a modified protocol for cytosolic and nuclear extract described previously (Emanuelli et al., J Biol Chem 275, 15985-91 (2000)). There was more FOXO1 protein translocated to the cytosol in shsirt2 3T3-L1 cells. SOD4 and LaminA (LmnA) bands showed effective separation of nuclear and cytosolic proteins. Immunocytochemistry was done with cells carrying FOXO1-FLAG overexpressing construct with either stably transfected shGFP or shsirt2. Cells were fixed 48 hours after being plated in 10% FBS DMEM media. The anti-FLAG-FITC was used to detect subcellular localization of the recombinant FOXO1 in the cells.

FIG. 10 is a set of images showing that FoxO1 knockdown in 3T3-L1 cells promotes adipocyte differentiation. Stable cell lines overexpressing shRNA targeting either endogenous FoxO1 or GFP were generated and subjected to white adipocyte differentiation protocol after 6 days. Total cell lysate was collected and subjected to SDS gel separation and western blotting with antibodies against different adipocyte markers. A marked increase in expression of PPAR and C/EBP in shFoxO1 cells after differentiation was seen, while cyclophillin A expression is unchanged between cell lines. The FoxO1 knockdown was detected using lysate from day 0 before cells were induced, western blotting showed about a 75% reduction endogenous FoxO1 protein expression in shFoxO1 cells as compared with shGFP cells. After 6 days of differentiation, cells were fixed and stained with Oil Red 0, more ORO staining in shFoxO1 cells indicated that these cells had more lipid accumulation than shGFP control cell line.

FIGS. 11A-11C are graphs and images showing that FOXO1 acetylation/deacetylation mimics regulate 3T3-L1 adipocyte differentiation and FOXO1 phosphorylation. FIG. 11A shows different foxo1 overexpression constructs made with either wild type FOXO1 amino acid sequence or replacing all three Lys residues surrounding Ser-253 with Glu (KQ) or Arg (KR). Thefoxo1 WT, KQ, and KR overexpression constructs were all FLAG tagged. Overexpression was determined by western blotting using anti-FLAG antibody. Quantitative PCR with primers targeting the foxo1 coding region showed that the level of overexpression of different constructs was similar and was about 5 times the level of endogenous foxo1 observed in control cells (FIG. 6). Cell lines carrying various foxo1 overexpression constructs or control cells were subjected to the differentiation protocol described below. Oil Red O staining of cells eight days after differentiation induction showed differences among different cell lines. PCR quantification of different adipocyte markers was consistent with the degree of adipocyte differentiation as accessed by bright light microscopic image and Oil Red O staining. Cells overexpressing wild type foxo1 had significantly decreased mRNA expression of different adipocyte differentiation markers comparing with control cells, as indicated with “a”; while cells expressing the KR mutant had significantly decreased mRNA expression compared with that of wild type foxo1 overexpression, as indicated with “b”. FIG. 11B shows FOXO1 mutations mimicking different Lys acetylation states affect Ser-253 phosphorylation of FOXO1 and 3T3-L1 adipocyte differentiation. After serum deprivation for 12 hours, 3T3-L1 cells carrying wild type foxo1, KQ, and KR mutant overexpression constructs were acutely stimulated with different concentrations (10 nM or 100 nM) of insulin for 10 minutes. Total cell extracts were subjected to western blot analysis to assess insulin stimulated phosphorylation status of FOXO1, Akt, and GSK3β. KQ mutant overexpression promoted both basal and insulin stimulated FOXO1 phosphorylation, whereas cells overexpressing the KR mutant had decreased FOXO1 phosphorylation in response to insulin (both as compared to cells overexpressing wild type foxo1). FIG. 11C shows immunocytochemistry of different foxo1 wild type and mutants overexpressing cells using anti-FLAG-FITC. Different subcellular localization patterns are observed for foxo1 mutants. Error bars in this figure refer to Standard Error (SE) of the mean.

FIG. 12 is a graph showing overexpression of recombinant FoxO1 wild type and mutants. Real-time PCR of FoxO1 indicated there was 5-fold increase of recombinant FoxO1 mRNA expression in different stable cell lines.

FIG. 13 is a list of amino acid sequences including isoforms of human sirtuin2 (SEQ ID NO:1 and SEQ ID NO:2), mouse Sir2 (SEQ ID NO:3), yeast Sir2 (SEQ ID NO:4), and Drosophila melanogaster Sirt2 (SEQ ID NO:5)

FIG. 14 is a list of amino acid sequences including human (SEQ ID NO:6) and mouse (SEQ ID NO:7).

### DETAILED DESCRIPTION

We have found that overexpression of sirtuin2 results in decreased adipogenesis. On this basis, increasing sirtuin2 expression or activity can be used for treating metabolic disorders such as obesity. In addition, sirtuin2 expression or activity levels may be indicative of a metabolic disorder or a propensity to develop such a disorder (e.g., obesity) in a subject.

From our studies, sirt2 mRNA is more abundant than other Sirts in both adipose tissue in vivo and preadipocytes in culture, with quantitative mRNA levels being four to seven times higher than those of Sirt1 or Sirt3. In addition, sirt2 and sirt1 expression is down regulated during adipocyte differentiation, whereas Sirt3 mRNA levels increase. In 3T3-L1 cell lines with stable overexpression and knockdown of sirt2, high levels of sirt2 expression can inhibit adipocyte differentiation, whereas reduction of sirt2 levels has the opposite effect. The promotion of adipocyte differentiation by sirt2 is associated with increased expression of C/EBPα, PPARγ, Glut4, aP2, and FAS mRNAs, as well as increased expression of C/EBPβ, one of the earliest transcriptional changes in the normal program of adipocyte differentiation (Tang et al., Biochem Biophys Res Commun 318, 213-8 (2004)). Thus, sirt2 must act upstream of C/EBPβ at an even earlier event in induction of adipogenesis, and this appears to be at the level of FOXO1 acetylation/phosphorylation. Reducing the level of sirt2 in the knockdown cells results in an increased level of FOXO1 acetylation, which in turn allows increased phosphorylation on Ser-253, excluding FOXO1 from the nucleus. This allows differentiation to progress, likely by reducing the ability of FOXO1 to interact with the PPARγ promoter and repress PPARγ transcription (Armoni et al., J Biol Chem 281, 19881-91 (2006)). Although there is evidence that foxo1 also acts during late stage differentiation, the effect of foxo1 over-expression on differentiation appear to occur prior to the induction of early differentiation markers like C/EBPβ/σ, possibly at the level of clonal expansion. The effect of sirt2 knockdown suggests that SIRT2 may act on foxo1 during this clonal expansion stage.

In the process of adipocyte differentiation, insulin and/or IGF-1 act to stimulate FOXO1 phosphorylation on Ser residues through activation of Akt. The Ser phosphorylation of FOXO1 excludes it from the nucleus (Zhang et al., J Biol Chem 277, 45276-84 (2002)), thus reducing its ability to repress PPARγ transcription. Changing the level of sirt2 alters the phosphorylation status of FOXO1, in this case not because of a change in insulin/IGF-1 action on Akt, but because phosphorylation of FOXO1 can also be regulated by acetylation/deacetylation of the Lys residues surrounding Ser-253, the major site of regulatory phosphorylation (Zhang et al., supra; Matsuzaki et al., Proc Natl Acad Sci USA 102, 11278-83 (2005)). While previous studies have suggested that CBP can act as a FOXO1 acetyl-transferase (Matsuzaki et al., supra; Perrot et al., Mol Endocrinol 19, 2283-98 (2005)), it is not clear which enzyme deacetylates FOXO1. In the nucleus, Sirt1 has been shown to deacetylate FOXO1. This increases the level of FOXO1 localized in the nucleus, allowing it to be transcriptionally active (Frescas et al., J Biol Chem 280, 20589-95 (2005)). In this study, we find that FOXO1 can also be a target of the cytoplasmic SIRT2 deacetylase, and that in this context sirt2 can play an important role in adipocyte differentiation.

This effect on differentiation appears to be a direct action of sirt2, rather than an indirect effect of sirt1. First, FOXO1 acetylation is increased by sirt2 knockdown and is independent of changes in levels of Sirt1 or foxo1 expression. Thus, SIRT2 likely deacetylates FOXO1, rather than acting indirectly by decreasing Sirt1 expression level. Second, SIRT2 interacts with FOXO1, as shown by co-immunoprecipitation experiments. Third, in sirt2 knockdown cells there is increased Ser-253 phosphorylation in response to insulin stimulation, which results in nuclear exclusion of FOXO1. This, in turn, releases adipogenesis from foxo1 inhibition. Acetylation of FOXO1 in the cytoplasm may thus increase its accessibility to Akt phosphorylation, which, in turn, promotes retention of FOXO1 in the cytosol where it is transcriptionally inactive. Increased cytoplasmic localization of FOXO1 renders it unable to repress expression of genes like PPARγ. In this way, increased acetylation reduces the inhibitory effect of foxo1 on adipogenesis, thereby promoting differentation.

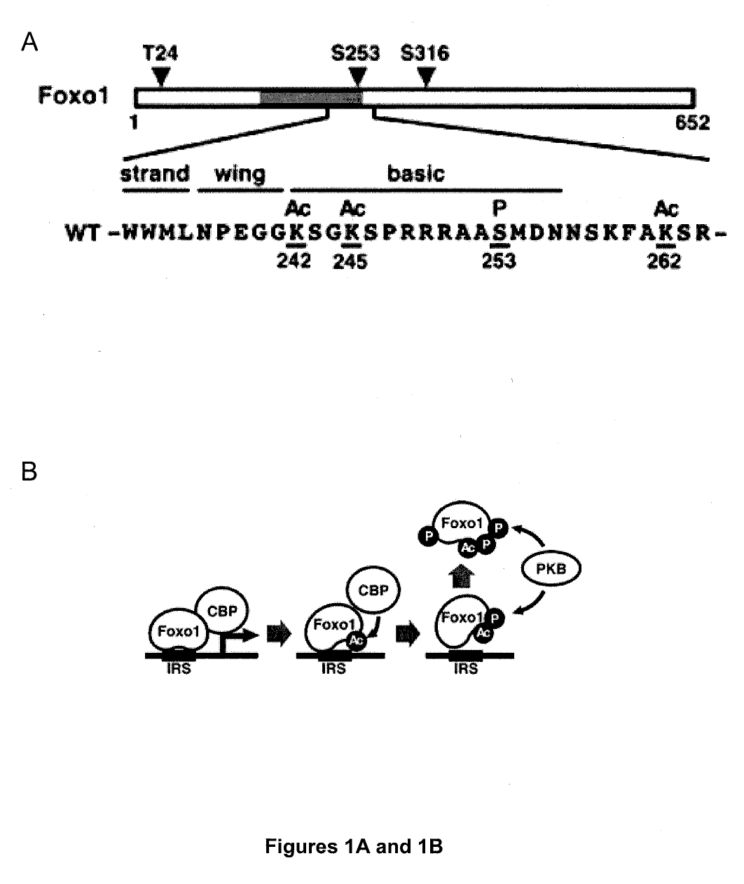

This role of acetylation of foxo1 in adipogenesis is further supported by our studies using foxo1 mutants described below. There are three Lys residues surrounding the Ser-253 in the wild type mouse FOXO1 protein (FIG. 1A). These three Lys residues can be acetylated by the protein acetyl-transferase CBP and can be deacetylated by Class III deacetylases, such as SIRT2 (FIG. 1B). Recent studies have shown that acetylation/deacetylation of these Lys and Ser phosphorylation can act in a synergistic manner (Matsuzaki et al., Proc Natl Acad Sci USA 102, 11278-83 (2005)). Thus when FOXO1 is acetylated by CBP, it is more accessible to phosphorylation, which leads to cytosolic translocation. In the foxo1 KQ mutant, the three Lys residues surrounding Ser-253 are replaced by Glu, mimicking a constitutive “acetylated” form of the protein. Previous studies on p53 have shown that substitution of Glu for Lys functions in a similar manner (Wang et al., J Biol Chem 278, 25568-76 (2003)). By contrast, replacing Lys with Arg, as in the foxo1 KR mutant, serves to mimic the “deacetylated” form of protein (Feng et al., Mol Cell Biol 25, 5389-95 (2005); Marcotte et al., Anal Biochem 332, 90-9 (2004)). In agreement with Matsuzaki et al. (Proc Natl Acad Sci USA 102, 11278-83 (2005)), we find that these two foxo1 mutants behave differently in terms of acetylation and Ser-253 phosphorylation in response to insulin stimulation when compared with wild type foxo1. Thus, overexpression of the KR mutant, which is acetylation resistant, inhibits 3T3-L1 differentiation to an even greater extent than wild type foxo1, whereas overexpression of the KQ mutant that mimics acetylated FOXO1 promotes differentiation. In each case, this correlates with the Ser phosphorylation of the FOXO1 protein. Cells overexpressing WT FOXO1 exhibit an increased level of Ser-253 phosphorylation following insulin activation of Akt, while cells expressing the KQ mutant have higher levels of FOXO1 phosphorylation with or without any insulin stimulation. Cells overexpressing the KR mutant demonstrate the opposite with reduced FOXO1 phosphorylation following insulin stimulation. Because all these occur with the same level of Akt and GSK3β phosphorylation/activation, these findings indicate that it is an intrinsic property of FOXO1 and its apparent acetylation status that modulates FOXO1 phosphorylation and adipocyte differentiation. In addition, changes of FOXO1 Lys residue acetylation can affect its DNA binding activity. The effects of foxo1 mutants on adipocyte differentiation may be mediated by similar changes. Combining these results with previous studies indicating that Sirt1 can also deacetylate FOXO1, SIRT2 may target FOXO1 in the cytoplasm, whereas Sirt1 may catalyze FOXO1 deacetylation in the nucleus. These two proteins may recruit different co-factors and have different physiological or pathological regulation allowing them to carry out distinctive functions on the target.

Because many of the Class III HDACs of the sirtuin family require NAD as a cofactor, the level of NAD may act as a regulator of sirt2 activity in normal cells. This would allow sirt2 to serve as a sensor of the cellular redox state and nutrient input with the ability to regulate gene expression and metabolism.

Transcriptional activation and repression in eukaryotic cells has been shown to be involved closely with protein acetylation/deacetylation mediated by histone acetyltransferases (HATs) and histone deacetylases (HDACs). The reversible acetyl-modification on Lys residues of transcription factors provides a mechanism by which modulating activities of either HATs or HDACs leads to changes in the expression of genes in metabolic pathways. This process may be further modulated by nutritional and redox state.

### The Sirtuin Family of Proteins

The Sir2 (silent information regulator 2) proteins belong to the family of class III NAD-dependent deacetylases that catalyze a reaction in which NAD and an acetylated substrate are converted into a deacetylated protein, nicotinamide and a novel metabolite O-acetyl ADP-ribose (Tanner et al., Proc Natl Acad Sci USA 97, 14178-82 (2000)). The founding member of the family, Sir2 was originally discovered in yeast as a factor that silences the mating type locus (Imai et al., Nature 403, 795-800 (2000); Tanny et al., Cell 99, 735-45 (1999)). Sir2 is also involved in telomere regulation, maintenance of genomic integrity and lifespan extension in yeast and similar effects have been shown for its orthologue in C. elegans (Imai et al., Nature 403, 795-800 (2000); Wang et al., Mech Ageing Dev 127, 48-56 (2006)).

In mammals, the homologues of Sir2 have been named sirtuins (sirt), with seven members in a family termed sirt1 through sirt7. They share a conserved central deacetylase domain, but have different N- and C-termini and display distinct subcellular localization suggesting different biological functions (North et al., Genome Biol 5, 224 (2004)). Mammalian sirt1 is most homologous to yeast sir2 and is found predominantly in the nucleus, consistent with its roles in formation of heterochromatin and gene silencing by histone deacetylation. In mammalian cells, instead of genome silencing, sirt1 often promotes gene transcription by deacetylating specific transcription factors, corepressors, and coactivators, including p53, PGC-1α, NF-kB, MyoD and members of the foxo family (Daitoku et al., Proc Natl Acad Sci USA 101, 10042-7 (2004); Fulco et al., Mol Cell 12, 51-62 (2003); Luo et al., Cell 107, 137-48 (2001); Nemoto et al., J Biol Chem 280, 16456-60 (2005); Yeung et al., EMBO J. 23, 2369-80 (2004)). In adipocytes, sirt1 acts as an inhibitor of adipogenesis by interacting with PPARγ co-repressor NcoR and SMART thereby repressing PPARγ activity (Picard et al., Nature 429, 771-6, (2004)).

In contrast to SIRT1, mammalian SIRT2 is localized mainly in the cytoplasm. Studies in mammalian cells suggest that sirt2 plays a role in cell cycle regulation and be involved in cytoskeleton organization by targeting the cytoskeletal protein tubulin (North et al., Mol Cell 11, 437-44 (2003)). The yeast ortholog of sirt2, hst2 has been shown to extend lifespan by a mechanism independent of sir2/hst1 (Lamming et al., Science 309, 1861-4, (2005)). SIRT3 deacetylates acetyl-CoA synthase 2 (ACS2) and regulates its activity (Hallows et al., Proc Natl Acad Sci USA 103, 10230-5 (2006)). sirt3 also appears to be involved in longevity (Rose et al., Exp Gerontol 38, 1065-70 (2003)). sirt6 may also be involved in aging in mice, while sirt7 appear to regulate DNA pol I transcription (Ford et al., Genes Dev 20, 1075-80 (2006); Mostoslavsky et al., Cell 124, 315-29 (2006)).

### FoxO Transcription Factors

Mammalian forkhead transcription factors of class O (FoxO) include foxO1, foxO3a, and foxO4 and are involved with cellular processes such as DNA repair, cell cycle control, stress resistance, apoptosis, and metabolism (Barthel et al., Trends Endocrinol Metab 16, 183-9 (2005); Furukawa-Hibi, et al., Antioxid Redox Signal 7, 752-60 (2005)). Foxo proteins are transcription factors that contain acetylation and phosphorylation sites that affect their transcription activity (FIG. 1A, which shows Foxo1). Regulation of Foxo proteins is mediated by CBP, which, in the case of Foxo1, initially induces transcriptional activity but subsequently decreases transcriptional activity by acetylation of Foxo1, as shown in FIG. 1B. Mouse silent information regulator 2 (sirtuin1) has been shown to potentiate Foxo1 transcriptional activity through deacetylation (Daitoku et al., (2004) Proc. Natl. Acad. Sci. USA 101, 10042-10047) and is involved in stress-dependent regulation of Foxo transcription factors. This deacetylation promotes expression of glucogenetic genes. Changes in the acetylation state of Foxo1 are shown to affect its DNA binding, as well as its sensitivity to phosphorylation (Matsuzaki et al., (2005) Proc. Natl. Acad. Sci. USA 102, 11278-11283).

Among all FoxO members, foxo1 appears to have an important role in adipocyte differentiation acting as an inhibitor of adipogenesis at an early phase of the differentiation process (Nakae Dev Cell 4, 119-29 (2003)). In this context, the enzyme phosphatidylinositol 3-kinase (PI-kinase), which is stimulated by insulin and certain cytokines and growth factors, can negatively regulate FoxOs (Zhang et al., J Biol Chem 277, 45276-84 (2002)). This inhibitory effect of insulin is mainly mediated by Akt/PKB phosphorylation of FoxO, which promotes the trafficking of FoxO from the nucleus to the cytosol. The transcriptional activity of FoxO proteins can also be regulated by acetylation and deacetylation. FOXO1 can be acetylated by CBP acetyl-transferase, and SIRT1 has been shown to deacetylate FOXO1 and regulate its activity, especially under conditions of stress (Matsuzaki et al., Proc Natl Acad Sci USA 102, 11278-83 (2005); van der Heide et al., Trends Biochem Sci 30, 81-6 (2005)). The extent of deacetylation of FOXO1 can affect its phosphorylation and DNA binding activity to target gene promoters (Matsuzaki et al., Proc Natl Acad Sci USA 102, 11278-83 (2005)).

### Sirt Isoform Expression During Adipocyte Differentiation of 3T3-L1 Cells

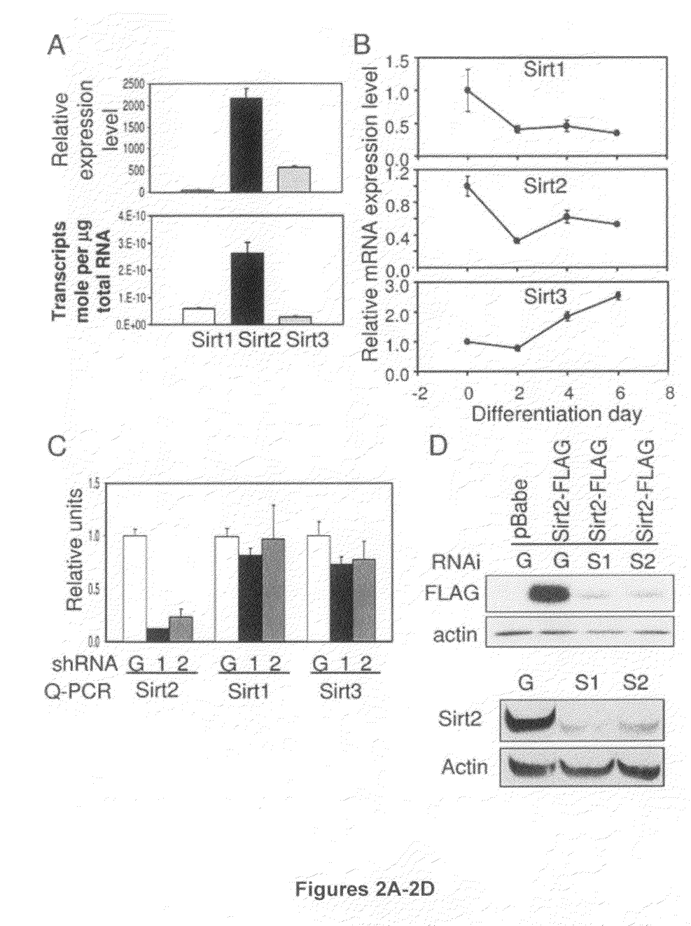

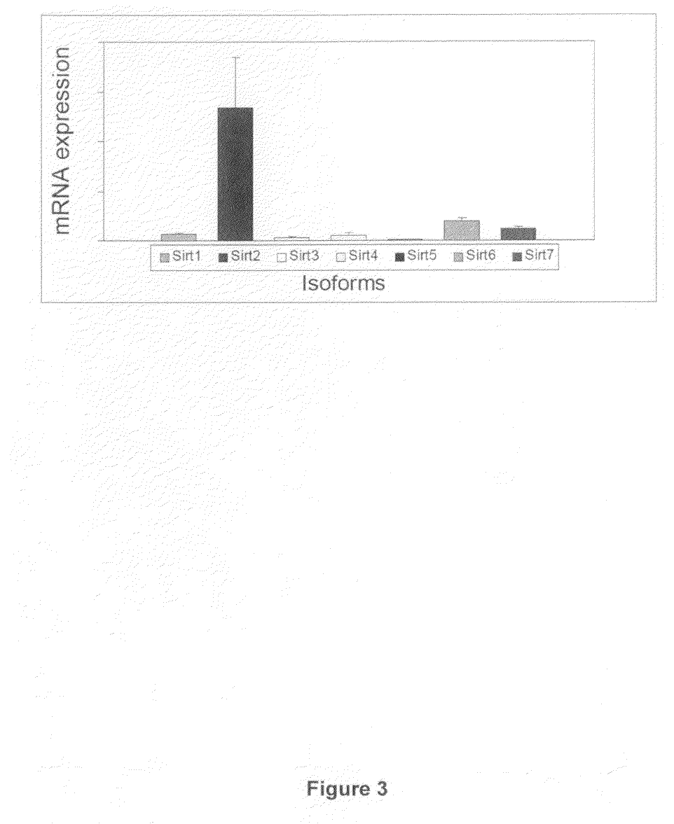

We studied the expression patterns of the different isoforms of mammalian Sirt proteins in adipose tissue and 3T3-L1 preadipocytes. The Sirt proteins exhibit different patterns during differentiation. Affymetrix microarray analysis performed on isolated adipocytes indicated expression of sirt1, sirt2, and sirt3 and that the level of sirt2 was much higher than that of sirt1 or sirt3 (FIG. 2A, top panel). Using quantitative real-time PCR with cDNA standard curves for each isoform, the molar amounts of different sirt transcripts per microgram of total RNA were obtained. As shown in FIG. 2A (lower panel), the molar amount of sirt2 mRNA per microgram total RNA in 3T3-L1 preadipocytes was 4-5 times that of sirt1 and 6-7 times of sirt3. Similar study including other sirt members confirmed that sirt2 transcripts are more abundant than others (FIG. 3). During the first 2 days of differentiation, i.e., the induction phase, levels of both sirt1 and sirt2 mRNA decreased by 60-70% and then remained stable for the remainder of the time course of differentiation (FIG. 2B, top and middle panels). Sirt3 mRNA on the other hand, started at a low level compared to both sirt2 and sirt1, then increased by 3-4 fold during adipocyte differentiation (FIG. 2B, bottom panel).

Effects of sirt2 Knockdown and Overexpression in 3T3-L1 Adipocytes

To investigate the potential role of sirt2 in preadipocytes, we used retroviruses to generate 3T3-L1 stable cell lines carrying either shRNAs targeting endogenous sirt2 or GFP as a control. We used real-time PCR to assess sirt2 mRNA levels and found that cells stably expressing the two shsirt2 retroviruses exhibited an 80-90% knockdown of sirt2 mRNA, with no significant change in the level of sirt1 or sirt3 mRNAs (FIG. 2C), compared with shGFP cells. When the same two retroviral constructs were transiently co-transfected into HEK293 with a CMV-driven SIRT2-FLAG construct, there was a parallel 80-90% reduction of the tagged SIRT2 protein when compared with cells co-transfected with shGFP (FIG. 2D). Thus, the expression of both shsirt2 constructs produced major reductions of sirt2 at the RNA and protein levels, and this reduction was specific to the sirt2 isoform. A similar decrease of endogenous SIRT2 protein in shsirt2 cells (FIG. 2D) was observed using a commercial availability SIRT2 antibody.

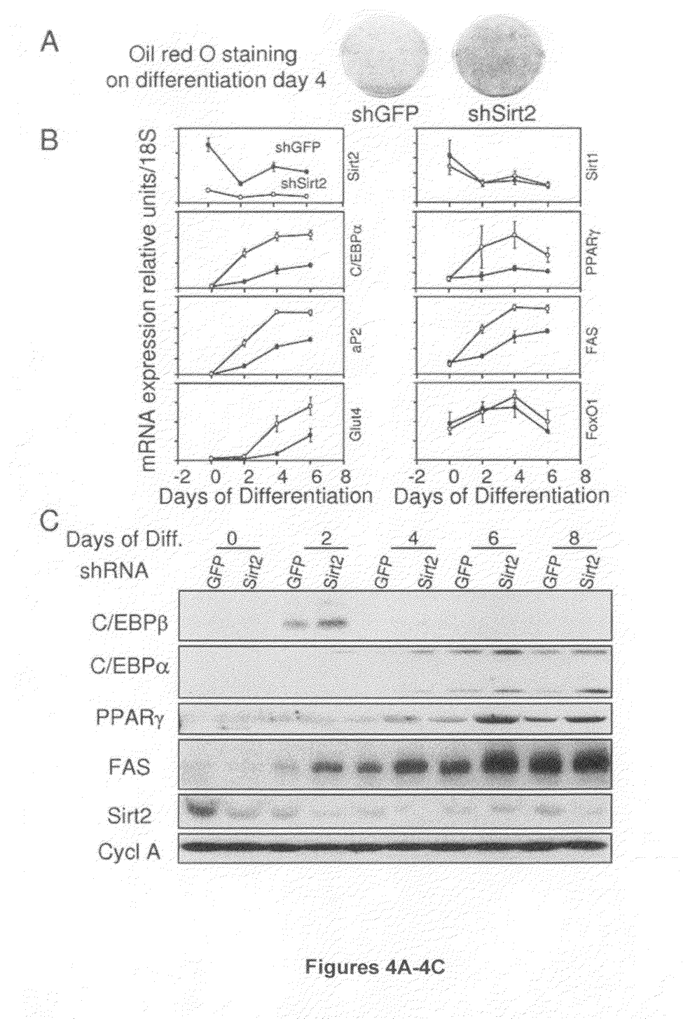

Pre-adipocytes stably transfected with either shsirt2 or shGFP were then subjected to an adipogenic differentiation protocol, and samples from different time points were collected for either RNA or protein analysis. Oil Red O staining during the time course of differentiation confirmed the increased rate and extent of differentiation with increased staining of cells by day 4, indicating more rapid accumulation of lipid in sirt2 knockdown cells (FIG. 4A). As noted above, in control shGFP-expressing cells sirt2 mRNA expression decreased during the time course of differentiation, while in the shsirt2 expressing cells, endogenous sirt2 mRNA as assessed by real-time PCR was reduced by 75-80%. This reduction persisted throughout the time course of adipocyte differentiation (FIG. 4B). As expected, sirt2 knockdown had no significant effect on levels of Sirt1 mRNA or on the change in Sirt1 that occurred during differentiation, consistent with the specificity of sirt2 knockdown (FIG. 4B). By contrast, in the sirt2 knockdown cells, two transcription factors central to adipogenic differentiation, C/EBPα and PPARγ, both demonstrated significantly accelerated and exaggerated increase in mRNA expression. Thus, C/EBPα mRNA level was elevated more than 3-fold in shsirt2 cells on day 2 after induction compared with control cells (P=0.001), and this difference remained throughout the time course of differentiation (FIG. 4B). PPARγ mRNA levels in shsirt2 cells were also 2- to 3-fold higher than in shGFP cells after induction and throughout time course with the greatest increase on day 2 (FIG. 4B). Corresponding to elevated early adipogenic transcription factor expression, mRNA levels of various late adipocyte differentiation markers that are downstream, C/EBPα and PPARγ (Lane et al., Biochem Biophys Res Commun 266, 677-83, (1999); Qi et al., Cell Biochem Biophys 32 Spring, 187-204 (2000)), were also significantly enhanced in shsirt2 cells during the time course of differentiation. For example, on day 2 after induction, shsirt2 cells had ˜3 fold higher levels of aP2 mRNA (P=0.002), and ˜2 fold higher levels of fatty acid synthase (FAS) (P=0.0014) and Glut 4 mRNA (P=0.0055) compared to control cells (FIG. 4B). Expression of FOXO1, showed no significant change at mRNA level at any time point during differentiation (FIG. 4B).

Western blot analysis of proteins confirmed the effects of sirt2 knockdown on expression of adipocyte differentiation markers (FIG. 4C). On day 2 after induction, there was a 2-fold increase in C/EBPβ and a 5-fold increase in C/EBPα protein in shsirt2 cells compared to control, and this increase in C/EBPα persisted through differentiation, even as levels in the control cells increased. A similar pattern of increased protein expression was observed for PPARγ protein in shsirt2 cells. The increase was even more marked for the late adipocyte differentiation marker, FAS, which was 4-fold elevated at the protein level in shsirt2 cells on day 2 compared to controls, although this difference diminished on day 8 as the cells became mature and FAS expression increased in the control cells (FIG. 4C). Endogenous SIRT2 protein expression was consistent with its mRNA expression during differentiation in shGFP cells, while endogenous SIRT2 protein was knocked down in shsirt2 cells.

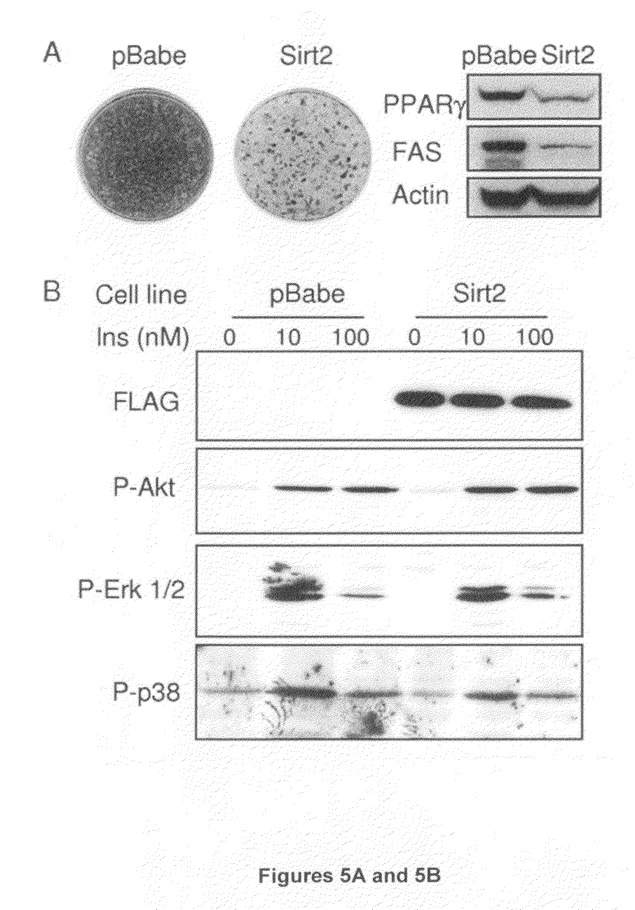

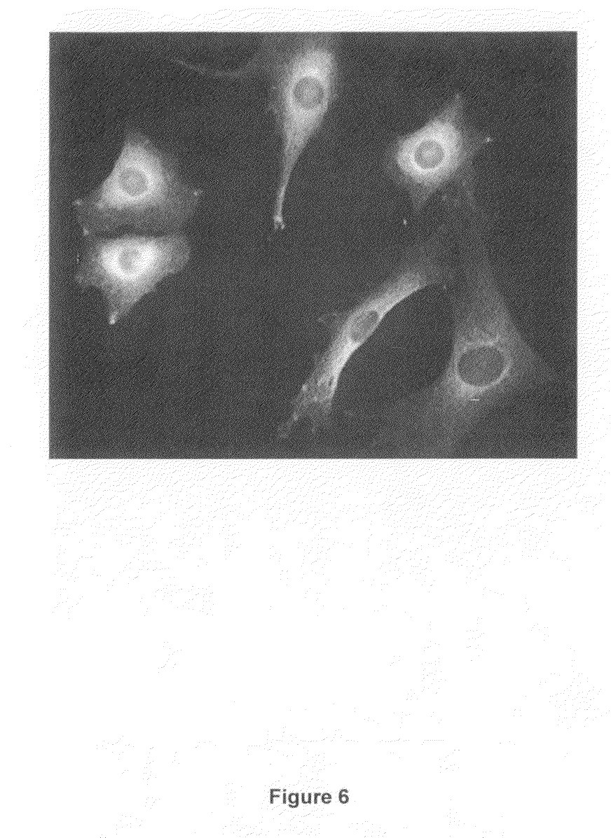

Opposite effects were observed in 3T3-L1 cells overexpressing sirt2. Over-expression of SIRT2-FLAG in 3T3-L1 cells inhibited adipocyte differentiation and lipid accumulation compared with empty vector control cells (FIG. 5A). Western blot analysis of adipocyte markers, such as PPARγ and FAS, also revealed decreased levels in sirt2 overexpressing cell line (FIG. 5A). As insulin signaling pathway is one of the major pathways that controls adipogenesis and adipocyte differentiation, we tested if the effect of sirt2 on 3T3-L1 differentiation was due to altered insulin signaling. Acute (10 minutes) insulin stimulation of both control and sirt2 overexpressing cell lines produced equal phosphorylation responses for Akt, p42/p44 MAP kinase and p38 MAP kinase (FIG. 5B). Thus, overexpression of sirt2 in 3T3-L1 cells inhibits the normal adipogenic process, and this effect occurs without a change in upstream insulin signaling. Conversely, reducing sirt2 expression enhanced the program of adipogenic gene expressions at the mRNA and protein levels, and this is associated with enhanced lipid accumulation. The subcellular localization of SIRT2-FLAG overexpression is similar to previous reports that SIRT2 is mainly a cytoplasmic protein (FIG. 6)

SIRT2 Interacts with and Deacetylates FOXO1 in 3T3-L1 Preadipocytes

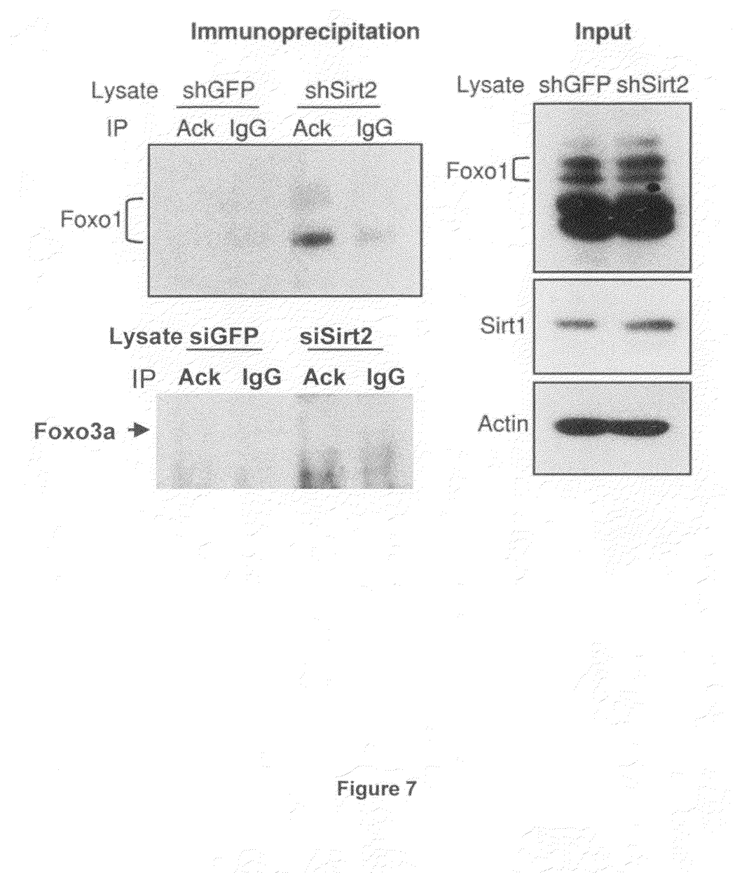

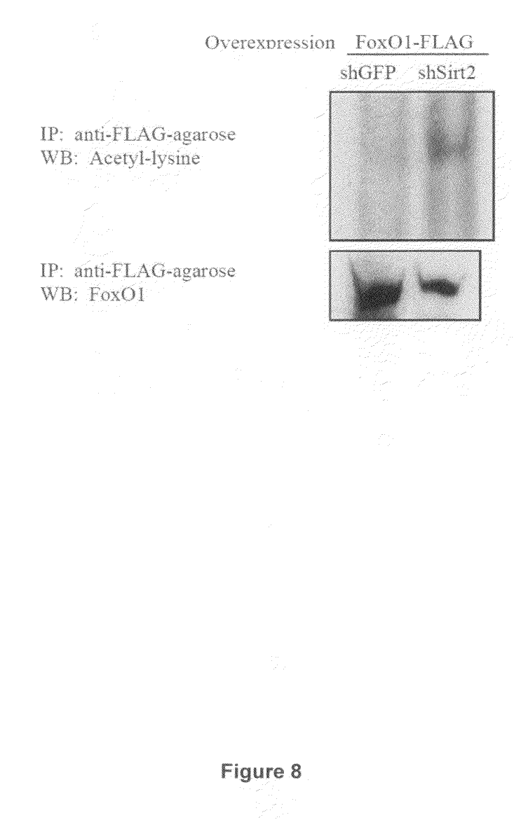

Foxo1, a known inhibitor of adipogenesis, undergoes regulated acetylation and deacetylation (Matsuzaki et al., Proc Natl Acad Sci USA 102, 11278-83 (2005); Perrot et al., Mol Endocrinol 19, 2283-98 (2005); Daitoku et al., Proc Natl Acad Sci USA 101, 10042-7 (2004)). Because there was no change in foxo1 expression at the mRNA level, we explored whether FOXO1 protein expression or acetylation might be changed. Immunoprecipitation using anti-acetyl-Lys antibody followed by blotting with anti-FOXO1 antibody revealed that in control shGFP cells, most of the FOXO1 protein was in a deacetylated state, i.e., FOXO1 could not be detected in precipitated total acetylated protein. By contrast, in the sirt2 knockdown cells, FOXO1 acetylation was markedly increased, and the anti-FOXO1 antibody easily detected the presence of FOXO1 protein in the precipitated lysate (FIG. 7). This effect was specific because western blot analysis with anti-FoxO3a antibody did not detect any increased protein acetylation (FIG. 7). To verify the increased FOXO1 acetylation in sirt2 knockdown cells, an immunoprecipitation of exogenous FOXO1-FLAG protein was done with shGFP and shsirt2 cell lysates. While a similar amount of FOXO1-FLAG protein was precipitated from either cell line, there was increased acetylation on FoxO1-FLAG precipitated from shsirt2 cell lysate (FIG. 8). These effects on FOXO1 acetylation occurred with no change in the total level of FOXO1 protein in sirt2 knockdown cells and no change in the level of Sirt1 protein (FIG. 7), another member of the Sirt family, which is able to deacetylate FoxOs.

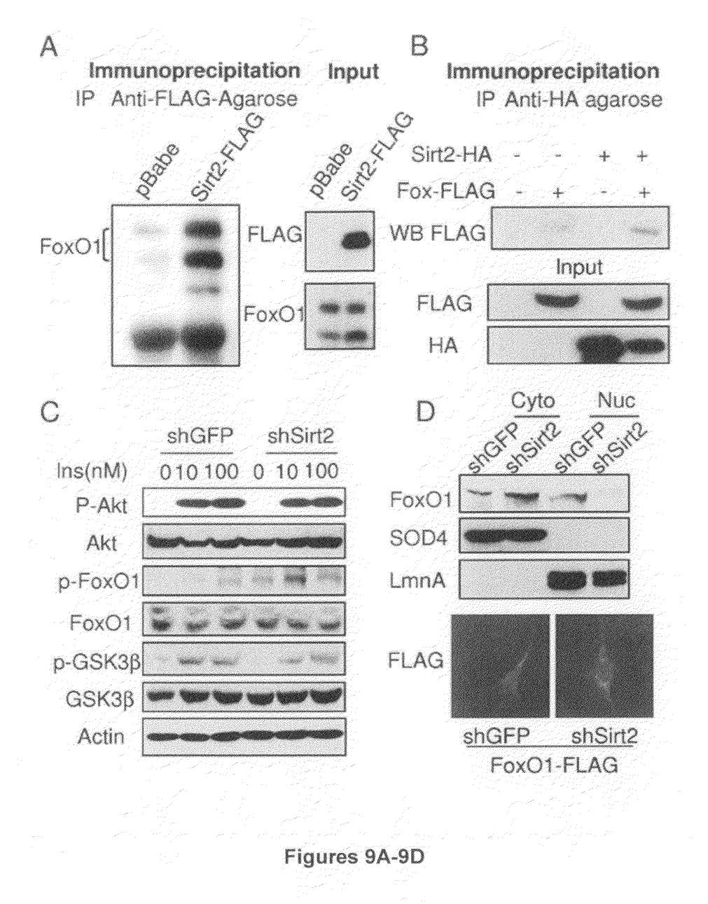

The increased acetylation on FOXO1 in sirt2 knockdown cells indicates that foxo1 can serve as a potential target for SIRT2 deacetylase activity. To investigate if SIRT2 interacts with FOXO1 directly, we performed immunoprecipitation of total cell lysates of cells overexpressing SIRT2-FLAG versus control cells infected with the empty pBabe retrovirus using a monoclonal anti-FLAG antibody conjugated to agarose. The immunoprecipitates were then immunoblotted with anti-FOXO1 antibody. In the cells expressing the SIRT2-FLAG construct, the anti-FLAG antibody co-precipitated significantly more FOXO1 protein than in control cells (FIG. 9A) indicating that SIRT2 is present in a complex with FOXO1 protein. Western blot analysis of the same cell lysates with anti-FOXO1 antibody showed that this occurred with no difference in total FOXO1 protein content between SIRT2-FLAG and control cell lines (FIG. 9A). The unchanged FOXO1 protein levels in preadipocytes of both sirt2 knockdown and overexpressing cells is consistent with the real-time PCR data indicating that foxo1 mRNA expression was not altered in sirt2 knockdown cells during 3T3-L1 differentiation (FIG. 4B). In lysates from cells overexpressing recombinant SIRT2-HA and FOXO1-FLAG, SIRT2-HA can co-immunoprecipitate FOXO1-FLAG in vitro (FIG. 9B), confirming the interaction between SIRT2 and FOXO1 protein.

### Acetylation of FOXO1 Regulates its Phosphorylation and Adipocyte Differentiation

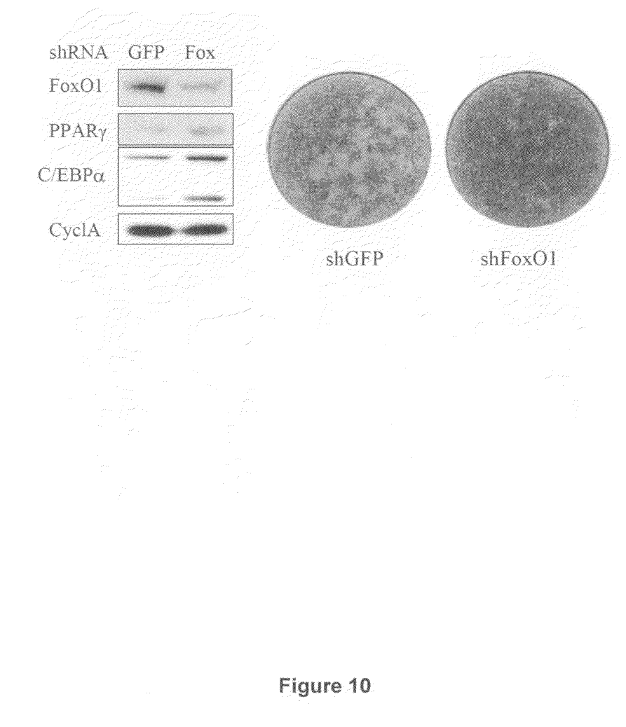

To determine if the increased acetylation of FOXO1 could alter its ability to undergo phosphorylation, we treated serum-deprived shsirt2 and shGFP preadipocytes with insulin at different concentrations and immunoblotted cell extracts with an antibody that detects phosphorylation of FOXO1 Ser-253, the major site of FOXO1 phosphorylation by Akt/PKB (van der Heide et al., Biochem J 380, 297-309 (2004)). Consistent with the data above, insulin stimulated Akt/PKB phosphorylation to the same level in the sirt2 knockdown and control cell lines. On the other hand, phosphorylation of FOXO1 at Ser-253 was increased two-fold in the sirt2 knockdown cell line (FIG. 9C). Because phosphorylation is known to affect nuclear translocation, nuclear and cytosolic extracts from shGFP and shsirt2 cells were prepared and subjected to immunoblot analysis with anti-FOXO1 antibody. This revealed a 2- to 3-fold increase in the level of cytosolic FOXO1 protein in sirt2 knockdown cells. Also, the cytosolic FOXO1 band migrated in a slightly retarded position on the gel in the shsirt2 cells, consistent with increased FOXO1 phosphorylation, whereas the nuclear FOXO1 protein migrated at a lower position on the gels due to its unphosphorylated state. Furthermore, nuclear FOXO1 was decreased in amount (FIG. 9D). Due to high background of FOXO1 antibody, we generated 3T3-L1 cell lines overexpressing FLAG tagged FOXO1, along with either shGFP or shsirt2 stable constructs, then used an anti-FLAG antibody to detect the subcellular localization of recombinant FOXO1. FOXO1-FLAG was largely excluded from nucleus of cells overexpressing shsirt2, while cells overexpressing shGFP showed more diffusive pattern of FOXO1-FLAG localization (FIG. 9D). Immunoblot of total protein lysates with anti-FOXO1 antibody revealed no difference in total FOXO1 protein between the two lines (FIG. 9C). To validate the effect of foxo1 on adipocyte differentiation, shRNA mediated endogenous foxo1 knockdown experiment was performed. From these experiments, reducing endogenous foxo1 expression efficiently promoted adipocyte differentiation with increased Oil RedO staining accompanied by increased adipocyte marker expression, including PPARγ and C/EBPα (FIG. 10).

### Analysis of FOXO1 Phosphorylation Using Acetylation Mutants

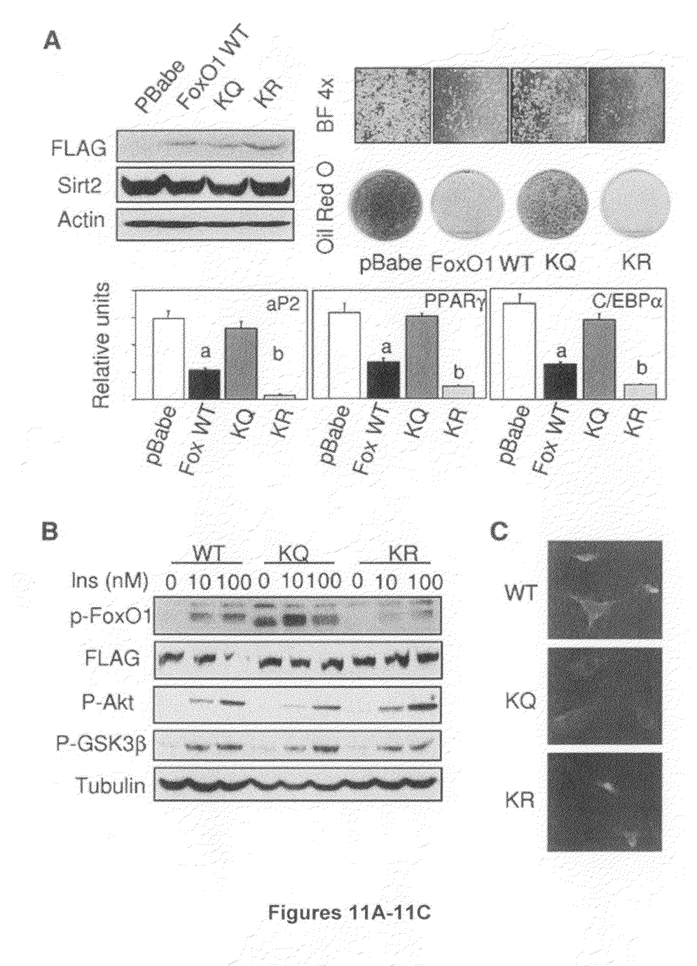

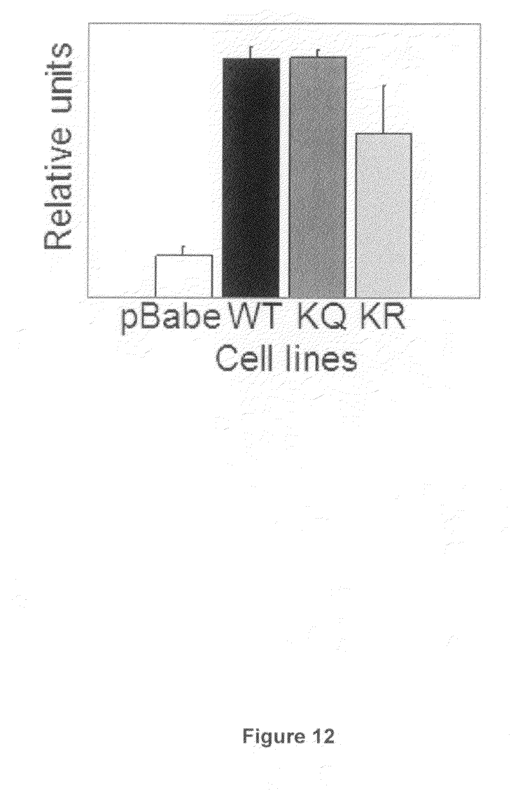

To further analyze the possible role of FOXO1 acetylation in regulation of FOXO1 phosphorylation, we used 3T3-L1 cell lines overexpressing either wild type foxo1 or two foxo1 mutants that mimic different acetylation states of the protein. In the KQ mutant, the three lysine residues surrounding Ser-253 known to be sites of acetylation, were replaced by glutamatic acid residues. In the KR mutant, these lysine residues were replaced by Arg residues. All three overexpression constructs were generated with a N-terminal FLAG tags to allow quantitation of the protein. Immunoblotting of lysates from confluent cells overexpressing either foxo1 wild type or the KQ and KR mutants with anti-FLAG monoclonal antibody revealed that all three proteins were equally overexpressed (FIG. 11A). Quantitative PCR indicated a 5-fold increase in total foxo1 mRNA in each line as compared to endogenous foxo1 levels (FIG. 12). Quantitative PCR using primers targeting the untranslated region of endogenous foxo1 mRNA demonstrated that the endogenous foxo1 expression level was not affected by expression of the exogenous protein (data not shown).

The cell lines overexpressing wild type and mutant foxo1 were subjected to the standard adipogenic differentiation protocol and stained with Oil Red 0. Cells overexpressing wild type foxo1 showed much less Oil Red O staining, consistent with a significantly decreased level of differentiation, than cells infected with the empty vector. This finding is consistent with known ability of foxo1 to suppress adipogenesis. The cells overexpressing the KQ mutant of foxo1, which mimics the acetylated state, exhibited enhanced differentiation compared with cells overexpressing wild type foxo1. In contrast, cells overexpressing the KR mutant, which mimics the deacetylated protein, showed decreased differentiation compared with cells overexpressing wild type foxo1 (FIG. 11A). These differences in lipid accumulation correlated well with expression of different adipocyte differentiation markers such as aP2, PPARγ, and C/EBPα by quantitative PCR (FIG. 11A).

Assessment of FOXO1 Ser-253 phosphorylation after insulin stimulation in these cell lines was performed. The experiments revealed increased phosphorylation of the KQ mutant in the basal state, as well as a substantially higher level of phosphorylation following insulin stimulation when compared with cells overexpressing wild type protein. By contrast, cells expressing the KR mutant of foxo1 showed decreased Ser-253 phosphorylation in the insulin-stimulated condition (FIG. 11B). Thus, the FOXO1 acetylation mimic had increased Ser-253 phosphorylation, whereas the deacetylated FOXO1 mimic had decreased Ser-253 phosphorylation. The FLAG western blot showed that the total recombinant FOXO1 protein expression is not altered under above conditions. These changes on FOXO1 phosphorylation occurred with no change in the level of phosphorylated/activated Akt and phosphorylated GSK3β (FIG. 11B). The subcellular localization of foxo1 mutants detected by immunocytochemistry using anti-FLAG-FITC was consistent with the observed differences in localization by sub-cellular fractionation and FOXO1 phosphorylation. Both foxo1 wild type and KR overexpression had a diffused distribution within the cell, but KQ mutant had a nuclear exclusion pattern, where the KQ was more phosphorylated and localized in the cytoplasm (FIG. 11B).

### Experimental Methods

The following methods were used in the experiments described above.

Cell Culture and Adipocyte Differentiation

HEK293 cells and 3T3-L1 (American Type Culture Collection, ATCC, Manassas, Va.) preadipocytes were cultured in high-glucose (400 mg/dl) Dulbecco's modified Eagle medium (DMEM, Invitrogen) containing 10% fetal bovine serum (FBS) (Gemini Bioproducts). 3T3-L1 cells, including different stable transfected cell lines used for differentiation, were maintained in 10% FBS DMEM with high glucose. Differentiation was induced 2 days after the cells reached confluence (day 0) by adding an induction cocktail containing 100 nM insulin (Sigma), 1 μM dexamethasone (Dex) (Sigma-Aldrich), and 0.5 mM 1-methyl-3-isobutyl-xanthine (IBMX) (Sigma-Aldrich) to the medium containing 10% FBS. After 2 additional days (day 2), the medium was replaced by DMEM 10% FBS containing 100 nM insulin, and then media was changed every 2 days until the cells became mature adipocytes (day 10). All cells were maintained and differentiated at 37° C. in an environment with 5% CO₂.

Plasmids and Constructs

For overexpression, a SIRT2-FLAG and SIRT2-HA construct was prepared using sirt2 cDNA derived from 3T3-L1 total cDNA produced by reverse transcription polymerase chain reaction, and inserted into pBabe-Bleo retroviral vector (Wei et al., Mol Cell Biol 23, 2859-70 (2003)). Sirt2 shRNAs were designed using the Dhamarcon website. Oligos containing sense and antisense siRNA sequence with separating loop region were synthesized by IDT DNA Technology Inc. Oligo pairs were annealed in a buffer containing 100 mM Tris HCL (pH 7.5), 1 M NaCl, and 10 mM EDTA, and then inserted into HindIII-BglII sites of pSuper-Retro vector (McIntyre et al., BMC Biotechnol 6, 1 (2006); Taxman et al., BMC Biotechnol 6, 7 (2006)). Oligonucleotide sequences are shown in Table 1 (SEQ ID NOS:8 and 9).

TABLE 1

| | |
|---|---|
| Oligo Name | Sequence |

| | |
|---|---|
| S-1 Forward | GATCCCCGAAGGAGTGACACGCTACAttcaagaga |
| | TGTAGCGTGTCACTCCTTCTTTTTGGAAA |
| S-1 Reverse | AGCTTTTCCAAAAAGAAGGAGTGACACGCTACAtctctt |
| | gaaTGTAGCGTGTCACTCCTTCGGG |

The FLAG tagged wild type foxo1, KQ (Lys residues converted to Glu) and KR (Lys residues converted to Arg) mutants cDNA were gifts from Dr. Akiyoshi Fukamizu of University of Tsukuba, Japan. Constructs of foxo1 wild type and mutants for overexpression were subcloned into pBabe bleo retroviral vectors.

Immunoprecipitation and Western Blot Analysis

For immunoprecipitation experiments, cells were grown to confluence, non-denaturing cell lysates were prepared and immunoprecipitation was done as previously described (Entingh et al., J Biol Chem 278, 33377-83, (2003)).

Western blot experiments were done after treatment and sample collection. Cell lysate was fractionated by SDS-10% polyacrylamide gel electrophoresis and transferred to PVDF membranes (Amersham). After blocking with recommended blocking reagents for 1 h at room temperature, the membranes were incubated overnight at 4° C. with different antibodies. Antibodies used for western blot and IP are shown in Table 2. The membranes were incubated with 1:2000-1:10000 secondary antibodies conjugated with HRP for 1 h at room temperature after washing for 10 minutes 3 times. Signals were detected by using the Amersham ECL chemiluminescence system and visualized by autoradiography.

TABLE 2

| | | Antibody | |
|---|---|---|---|
| Antibody Target | Dilution | Type | Vendor |
| anti-FLAG M2 | 1:10000 | Mouse | Sigma |
| anti-actin | 1:5000 | Rabbit | Santa Cruz |
| anti-PPARγ | 1:1000 | Rabbit | Upstate |
| C/EBPβ | 1:1000 | Rabbit | Santa Cruz |
| C/EBPα | 1:1000 | Rabbit | Santa Cruz |
| FAS | 1:2000 | Rabbit | Abcam |
| Glut4 | 1:1000 | Rabbit | Chemicon |
| SOD4 | 1:1000 | Rabbit | Abcam |
| Lamin A | 1:1000 | Rabbit | Abcam |
| FoxO1 | 1:1000 | Rabbit | Santa Cruz |
| Ser-253 phosphorylated FoxO1 | 1:1000 | Rabbit | Cell Signaling |
| Sirt2 | 1:1000 | Rabbit | Cell signaling |
| Sirt1 | 1:2000 | Rabbit | Upstate |
| Akt | 1:1000 | Rabbit | Cell Signaling |
| MAPK | 1:1000 | Rabbit | Cell Signaling |
| Phosphorylated Akt Ser307 | 1:1000 | Rabbit | Cell Signaling |
| Phosphorylated MAPK | 1:1000 | Rabbit | Cell Signaling |
| Phosphorylated p38 MAPK | 1:1000 | Rabbit | Cell Signaling |
| Acetylated lysine | 1:1000 | Mouse | Upstate |

Retroviral Infection and Transient Transfection

Retroviruses were produced as previously described (Entingh et al., J Biol Chem 278, 33377-83, (2003)). Stable retroviral transduction of 3T3-L1 cells was achieved by infection for 12-16 hours. The cells were plated into 30 cm diameter Petri dishes and grown for 48-72 hours, after which selection with either Puromycin (2 μg/ml) or Zeocin (250 μg/ml) was initiated. Selection was stopped as soon as the non-infected control cell died off, and the media was replaced with normal growing media. The efficacy of overexpression was determined by western blot. The efficacy of knockdown at the protein level was assessed using both western blots.

The co-transfection for recombinant SIRT2-HA and FOXO1-FLAG was done in HEK293 cells using Lipofectamine2000 (Invitrogen).

Immunocytochemistry

After grown on coverslips for 48 hours in 10% FBS DMEM media, cells were fixed with 10% formalin, washed with PBS 3 times, and permeablized with 1% TritonX 100 and 1% BSA in PBS. After washing 3 times, fixed cells were blocked with 10% goat serum and 1% BSA for 1 hour, then incubated with FLAG-conjugate antibody in 1% BSA for 1-2 hours. Signal was detected using a GFP fluorescent microscope.

Quantitative PCR

RNA samples were extracted using RNeasy kit (Qiagen). Each condition was performed in triplicate to allow for statistical analysis. The cDNA was synthesized using 1 μg total RNA using All Advantage RT-PCR kit. For quantification of relative expression levels of different Sirt mRNAs, 5 μl of cDNA was used for each reaction. To quantify the molar amount of RNA present in the samples, end product of real-time PCR for different Sirt genes were purified with PCR MiniElute kit (Qiagen), then quantified with NanoDrop 1000 and serially diluted 10-fold for each product, quantitative real-time PCR was performed using diluted PCR products with corresponding primers, Ct values of different dilutions were obtained, and linear regression graphs were created for each gene with absolute units derived from Ct values and corresponding molar amount based on PCR sizes. The corresponding target transcript molar amount used in Quantitative real-time PCR was accessed from the linear regression, then the molar amount of each gene per microgram total RNA was calculated based on total cDNA synthesis reaction volume and cDNA volume used for real-time PCR. For the differentiation time course experiments, realtime PCR was performed with 5 μl of cDNA using Sybrgreen master mix (Applied Biosystems) on ABI 7000 thermal cycler, and dCt values were collected by using either 18S ribosomal RNA or TATA-box binding protein (TBP) to normalize expression. The dCt values were calculated using absolute Ct values of the normalizer subtracted by Ct values of target genes. Final values were calculated using 2 exponential to the −dCt. Student t-test was performed between two different cell lines and significance was achieved when P<0.05. Primers for real-time PCR using Sybrgreen are shown in Table 3. Microarray data set generated using mRNA purified from isolated intra-abdominal adipocytes have been deposited in NCBIs Gene Expression Omnibus (GEO, http://www.ncbi.nlm.nih.gov/geo/) and are accessible through GEO Series accession number GSE8505 (GSM210983, GSM210984, GSM210985).

TABLE 3

Primers (SEQ ID NOS:10-37)

| | | |
|---|---|---|
| Gene Name | Forward Primer Sequence 5′-3′ | Reverse Primer Sequence 5′-3′ |

| | | |
|---|---|---|
| Glut4 | TGATTCTGCTGCCCTTCTGT | GGACATTGGACGCTCTCTCT |
| C/EBPα | CAAGAACAGCAACGAGTACCG | GTCACTGGTCAACTCCAGCAC |
| FoxO1 | GCTTTTGTCACATGCAGGT | CGCACAGAGCACTCCATAAA |
| FABP4/aP2 | GATGCCTTTGTGGGAACCT | CTGTCGTCTGCGGTGATTT |
| TBP | ACCCTTCACCAATGACTCCTATG | TGACTGCAGCAAATCGCTTGG |
| FAS | GGCTCTATGGATTACCCAAGC | CCAGTGTTCGTTCCTCGGA |
| PPARγ | TCAGCTCTGTGGACCTCTCC | ACCCTTGCATCCTTCACAAG |
| Sirt1 | AGAACCACCAAAGCGGAAA | TCCCACAGGAGACAGAAACC |
| Sirt2 | AGCCAACCATCTGCCACTAC | CCAGCCCATCGTGTATTCTT |
| Sirt3 | TGCTACTCATCTTGGGACCT | CACCAGCCTTTCCACACC |
| Sirt4 | GAGCAACTGGGAGAGACTGG | ACAGCACGGGACCTGAAA |
| Sirt5 | CGCTGGAGGTTACTGGAGA | CGTCAATGTTCTGGGTGATG |
| Sirt6 | CATGGGCTTCCTCAGCTTC | AACGAGTCCTCCCAGTCCA |
| Sirt7 | AGCCTACCCTCACCCACA | CGCTCAGTCACATCAAACAC |

### Diagnostic Assays

On the basis of the relationship identified between sirtuin2 and adipocyte differentiation, the present invention provides assays useful in the diagnosis of metabolic disorders such as obesity and diabetes, based on the discovery that sirtuin2 decreases adipocyte differentiation. Accordingly, diagnosis of metabolic disorders can be performed by measuring the level of expression or activity of sirtuin2 in a sample taken from a subject. This level of expression or activity can then be compared to a control sample, for example, a sample taken from a control subject, and a decrease in sirtuin2 expression or activity, relative to the control, is taken as diagnostic of a metabolic disorder, or an increased risk of or propensity to develop a metabolic disorder.

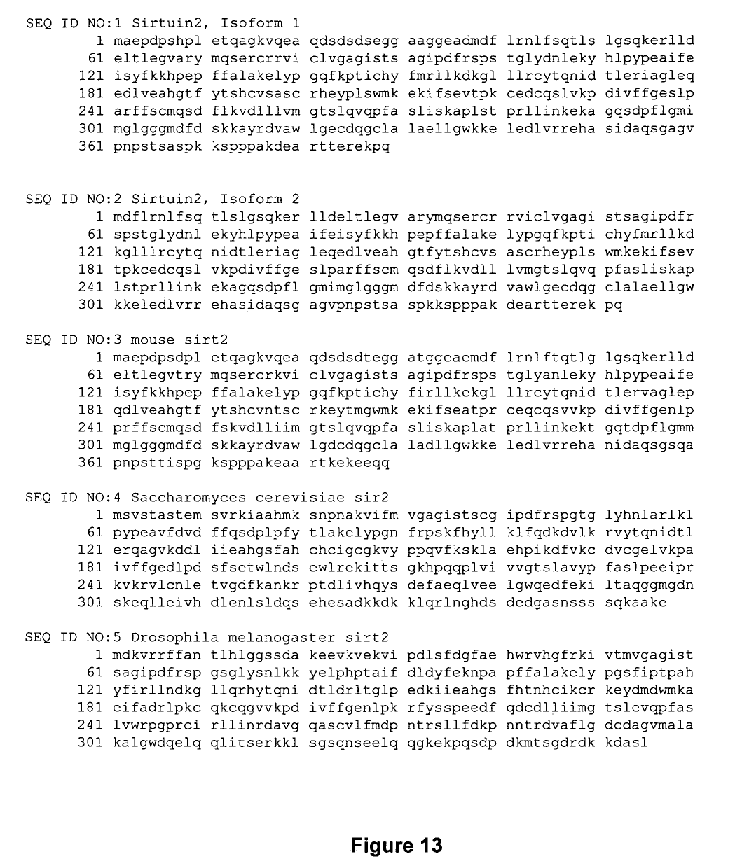

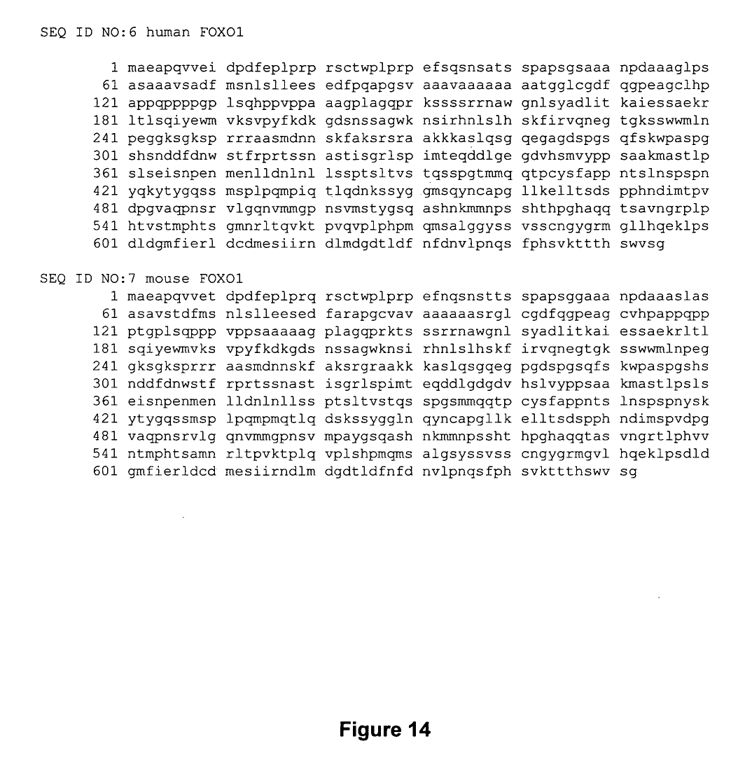

Analysis of levels of sirtuin2 mRNA or polypeptide, or activity of the polypeptide, may be used as the basis for screening the subject sample (e.g., a blood or tissue sample). Sirtuin2 nucleic acid and amino acid sequences are available in the art. For example, the nucleic acid amino acid sequences of human sirtuin2 are provided, for example, in Genbank accession numbers NM_{—}012237, and NM_{—}030593; coding sequences are shown as SEQ ID NO:1 and SEQ ID NO:2 (FIG. 13). Methods for screening mRNA levels include any of those standard in the art, for example, Northern blotting. Methods for screening polypeptide levels may include immunological techniques standard in the art (e.g., western blot or ELISA), or may be performed using chromatographic or other protein purification techniques. In another embodiment, the activity (e.g., histone deactelyase activity) of sirtuin2 may be measured, where a decrease in sirtuin2 activity relative to sample taken from a control subject is diagnostic of the metabolic disorder. Such activity may be measured by any standard prior art method, for example, the method described by Yoshida et al. (J. Biol. Chem. 265, 17174-17179 (1990)).

### Screening Methods to Identify Candidate Therapeutic Compounds

The invention also provides screening methods for the identification of compounds that bind to or modulate expression or activity of sirtuin2 and thus may be useful in the treatment of metabolic disorders such as diabetes or obesity. Useful compounds increase the expression or activity of sirtuin2.

Screening Assays

Screening assays to identify compounds that increase the expression or activity of sirtuin2 (e.g., increased binding to or deacetylation of Foxo1) are carried out by standard methods. The screening methods may involve high-throughput techniques. In addition, these screening techniques may be carried out in cultured cells or in organisms such as worms, flies, or yeast. Screening in these organisms may include the use of polynucleotides homologous to human sirtuin2. For example, a screen in yeast may include measuring the effect of candidate compounds on expression or activity of the yeast Sir2 gene (which encodes the yeast Sir2 polypeptide (SEQ ID NO:4)), or a screen in flies may include measuring the effect of candidate compounds on the expression levels or activity of the Drosophila melanogaster Sirt2 gene or Sirt2 polypeptide (SEQ ID NO:5).

Any number of methods is available for carrying out such screening assays. According to one approach, candidate compounds are added at varying concentrations to the culture medium of cells expressing a polynucleotide coding for sirtuin2. Gene expression is then measured, for example, by standard Northern blot analysis (Ausubel et al., Current Protocols in Molecular Biology, Wiley Interscience, New York, 1997), using any appropriate fragment prepared from the polynucleotide molecule as a hybridization probe. The level of gene expression in the presence of the candidate compound is compared to the level measured in a control culture medium lacking the candidate molecule. A compound which promotes an increase in sirtuin2 expression is considered useful in the invention; such a molecule may be used, for example, as a therapeutic for a metabolic disorder (e.g., obesity).

If desired, the effect of candidate compounds may, in the alternative, be measured at the level of polypeptide production using the same general approach and standard immunological techniques, such as western blotting or immunoprecipitation with an antibody specific for sirtuin2. For example, immunoassays may be used to detect or monitor the expression of sirtuin2. Polyclonal or monoclonal antibodies which are capable of binding to such a polypeptide may be used in any standard immunoassay format (e.g., ELISA, western blot, or RIA assay) to measure the level of sirtuin2. A compound which promotes an increase in the expression of the sirtuin2 is considered particularly useful. Again, such a molecule may be used, for example, as a therapeutic for a metabolic disorder (e.g., obesity).

Alternatively, or in addition, candidate compounds may be screened for those which specifically bind to and activate sirtuin2. The efficacy of such a candidate compound is dependent upon its ability to interact with the polypeptide. Such an interaction can be readily assayed using any number of standard binding techniques and functional assays (e.g., those described in Ausubel et al., supra). For example, a candidate compound may be tested in vitro for interaction and binding with sirtuin2 and its ability to modulate its activity may be assayed by any standard assays (e.g., those described herein).

In one embodiment, candidate compounds that affect binding of sirtuin2 to FOXO1 or deacetylation of Foxo1 by sirtuin2 are identified. Disruption by a candidate compound of sirtuin2 binding to Foxo1 may be assayed using methods standard in the art. The acetylation state of FOXO1 may, for example, be assayed using an antibody to acetylated lysine (e.g., the Ack antibody), as described herein. Compounds that affect binding of sirtuin2 to Foxo1 or affect the deacetylation of Foxo1 by sirtuin2 are considered compounds useful in the invention. Such compound may be used, for example, as a therapeutic in a metabolic disorder (e.g., obesity and diabetes).

In one particular embodiment, a candidate compound that binds to sirtuin2 may be identified using a chromatography-based technique. For example, recombinant sirtuin2 may be purified by standard techniques from cells engineered to express sirtuin2 and may be immobilized on a column. A solution of candidate compounds is then passed through the column, and a compound specific for sirtuin2 is identified on the basis of its ability to bind to the polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). Compounds isolated by this approach may also be used, for example, as therapeutics to treat a metabolic disorder (e.g., diabetes and obesity). Compounds which are identified as binding to sirtuin2 with an affinity constant less than or equal to 10 mM are considered particularly useful in the invention.

Potential agonists and antagonists include organic molecules, peptides, peptide mimetics, polypeptides, and antibodies that bind to sirtuin2, or a polynucleotide encoding sirtuin2 and thereby increase its activity. Alternatively, small molecules may act as agonists and bind sirtuin2 such that its activity is increased.

Polynucleotide sequences coding for sirtuin2 may also be used in the discovery and development of compounds to treat metabolic disorders (e.g., diabetes and obesity). Sirtuin2, upon expression, can be used as a target for the screening of drugs. Additionally, the polynucleotide sequences encoding the amino terminal regions of the encoded polypeptide or Shine-Delgarno or other translation facilitating sequences of the respective mRNA can be used to construct antisense sequences to control the expression of the coding sequence of interest. Polynucleotides encoding fragments of sirtuin2 may, for example, be expressed such that RNA interference takes place, thereby reducing expression or activity of sirtuin2.

The antagonists and agonists of the invention may be employed, for instance, to treat a variety of metabolic disorders such as diabetes and obesity.

Optionally, compounds identified in any of the above-described assays may be confirmed as useful in delaying or ameliorating metabolic disorders in either standard tissue culture methods or animal models and, if successful, may be used as therapeutics for treating metabolic disorders.

Small molecules provide useful candidate therapeutics. Preferably, such molecules have a molecular weight below 2,000 daltons, more preferably between 300 and 1,000 daltons, and most preferably between 400 and 700 daltons. It is preferred that these small molecules are organic molecules.

Test Compounds and Extracts

In general, compounds capable of treating a metabolic disorder (e.g., obesity and diabetes) are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and polynucleotide-based compounds. Synthetic compound libraries are commercially available. Alternatively, libraries of natural compounds in the form of bacterial, fungal, plant, and animal extracts are commercially available. In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity in treating metabolic disorders should be employed whenever possible.

When a crude extract is found to have an activity that increases sirtuin2 expression or activity, or a binding activity, further fractionation of the positive lead extract is necessary to isolate chemical constituents responsible for the observed effect. Thus, the goal of the extraction, fractionation, and purification process is the characterization and identification of a chemical entity within the crude extract having activity that may be useful in treating a metabolic disorder (e.g., diabetes and obesity). Methods of fractionation and purification of such heterogenous extracts are known in the art. If desired, compounds shown to be useful agents for the treatment of a metabolic disorder (e.g., obesity and diabetes) are chemically modified according to methods known in the art.

### Treatment of a Metabolic Disorder

The invention also provides methods for treating metabolic disorders such as diabetes and obesity by administration of a compound that increases expression or activity of sirtuin2 in a subject. The compounds used in the treatment of metabolic disorders may, for example, be compounds identified using the screening methods described herein.

Sirtuin2

Treatment of a subject with a metabolic disorder such as obesity may be achieved by administration of sirtuin2, or a fragment thereof having biological activity. Administration may be by any route described herein; however, parenteral administration is preferred. Additionally, the sirtuin2 polypeptide administered may include modifications such as post-translational modifications (e.g., glycosylation, phosphorylation), or other chemical modifications, for example, modifications designed to alter distribution of sirtuin2 within the subject or alter rates of degradation and/or excretion of sirtuin2.

Resveratrol and Derivatives

Resveratrol, a chemical found in grapes and other plants, has been observed to activate sirtuin2 (Suzuki et al., Biochem Biophys Res Commun. 359, 665-71 (2007)). Resveratrol and its derivatives may thus be used in the methods of the invention. Exemplary derivatives of resveratrol are described in PCT Publication No. WO 99/59561, hereby incorporated by reference.

Gene Therapy

Increases in sirtuin2 expression or activity may also be achieved through introduction of gene vectors into a subject. To treat a metabolic disorder such as obesity, sirtuin2 expression may be increased, for example, by administering to a subject a vector containing a polynucleotide sequence encoding sirtuin2, operably linked to a promoter capable of driving expression in targeted cells. In another approach, a polynucleotide sequence encoding a protein that increases transcription of the sirtuin2 gene may be administered to a subject with a metabolic disorder. Any standard gene therapy vector and methodology may be employed for such administration.

### Formulation of Pharmaceutical Compositions

The administration of any composition described herein (e.g., sirtuin2 or a sirtuin2 expression vector) or identified using the methods of the invention may be by any suitable means that results in a concentration of the compound that treats a metabolic disorder. The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously or intramuscularly), rectal, cutaneous, nasal, vaginal, inhalant, skin (patch), ocular, or intracranial administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions may be formulated to release the active compound immediately upon administration or at any predetermined time or time period after administration. The latter types of compositions are generally known as controlled release formulations, which include (i) formulations that create substantially constant concentrations of the agent(s) of the invention within the body over an extended period of time; (ii) formulations that after a predetermined lag time create substantially constant concentrations of the agents of the invention within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s) (sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the compound to a particular target cell type. Administration of the compound in the form of a controlled release formulation is especially preferred for compounds having a narrow absorption window in the gastro-intestinal tract or a relatively short biological half-life.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the compound is formulated with appropriate excipients into a pharmaceutical composition that, upon administration, releases the compound in a controlled manner. Examples include single or multiple unit tablet or capsule compositions, oil solutions, suspensions, emulsions, microcapsules, molecular complexes, microspheres, nanoparticles, patches, and liposomes.

### Parenteral Compositions

The composition containing compounds described herein or identified using the methods of the invention may be administered parenterally by injection, infusion, or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the active agent(s), the composition may include suitable parenterally acceptable carriers and/or excipients. The active agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

As indicated above, the pharmaceutical compositions according to the invention may be in a form suitable for sterile injection. To prepare such a composition, the suitable active agent(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, dextrose solution, and isotonic sodium chloride solution. The aqueous formulation may also contain one or more preservatives (e.g., methyl, ethyl, or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in the form of aqueous suspensions, microspheres, microcapsules, magnetic microspheres, oil solutions, oil suspensions, or emulsions. The composition may also be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutamine), poly(lactic acid), polyglycolic acid, and mixtures thereof. Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters)) or combinations thereof.

### Solid Dosage Forms for Oral Use

Formulations for oral use include tablets containing the active ingredient(s) in a mixture with non-toxic pharmaceutically acceptable excipients, and such formulations are known to the skilled artisan (e.g., U.S. Pat. Nos. 5,817,307, 5,824,300, 5,830,456, 5,846,526, 5,882,640, 5,910,304, 6,036,949, 6,036,949, 6,372,218, hereby incorporated by reference). These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and anti-adhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the compound in a predetermined pattern (e.g., in order to achieve a controlled release formulation) or it may be adapted not to release the agent(s) until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols, and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate, may be employed.

The solid tablet compositions may include a coating adapted to protect the composition from unwanted chemical changes, (e.g., chemical degradation prior to the release of the active substances). The coating may be applied on the solid dosage form in a similar manner as that described in Encyclopedia of Pharmaceutical Technology, supra.

The compositions of the invention may be mixed together in the tablet, or may be partitioned. In one example, a first agent is contained on the inside of the tablet, and a second agent is on the outside, such that a substantial portion of the second agent is released prior to the release of the first agent.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate, or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus, or spray drying equipment.

### Controlled Release Oral Dosage Forms

Controlled release compositions for oral use may, e.g., be constructed to release the compound by controlling the dissolution and/or the diffusion of the compound.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, DL-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax, and stearyl alcohol, carbopol 934, silicone, glyceryl tristearate, methyl acrylate-methyl methacrylate, polyvinyl chloride, polyethylene, and/or halogenated fluorocarbon.

A controlled release composition containing compounds described herein or identified using methods of the invention may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the composition with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Dosages

The dosage of any compound described herein or identified using the methods described herein depends on several factors, including: the administration method, the metabolic disorder to be treated, the severity of the metabolic disorder, whether the metabolic disorder is to be treated or prevented, and the age, weight, and health of the subject to be treated.

With respect to the treatment methods of the invention, it is not intended that the administration of a compound to a subject be limited to a particular mode of administration, dosage, or frequency of dosing; the present invention contemplates all modes of administration, including intramuscular, intravenous, intraperitoneal, intravesicular, intraarticular, intralesional, subcutaneous, or any other route sufficient to provide a dose adequate to treat hepatitis. The compound may be administered to the subject in a single dose or in multiple doses. For example, a compound described herein or identified using screening methods of the invention may be administered once a week for, e.g., 2, 3, 4, 5, 6, 7, 8, 10, 15, 20, or more weeks. It is to be understood that, for any particular subject, specific dosage regimes should be adjusted over time according to the individual need and the professional judgment of the person administering or supervising the administration of the compound. For example, the dosage of a compound can be increased if the lower dose does not provide sufficient activity in the treatment of a metabolic disorder (e.g., diabetes or obesity). Conversely, the dosage of the compound can be decreased if the metabolic disorder is reduced or eliminated.

While the attending physician ultimately will decide the appropriate amount and dosage regimen, a therapeutically effective amount of a compound described herein (e.g., histone deacetylase inhibitors) or identified using the screening methods of the invention, may be, for example, in the range of 0.0035 μg to 20 μg/kg body weight/day or 0.010 μg to 140 μg/kg body weight/week. Desirably a therapeutically effective amount is in the range of 0.025 μg to 10 μg/kg, for example, at least 0.025, 0.035, 0.05, 0.075, 0.1, 0.25, 0.5, 1.0, 1.5, 2.0, 2.5, 3.0, 3.5, 4.0, 5.0, 6.0, 7.0, 8.0, or 9.0 μg/kg body weight administered daily, every other day, or twice a week. In addition, a therapeutically effective amount may be in the range of 0.05 μg to 20 μg/kg, for example, at least 0.05, 0.7, 0.15, 0.2, 1.0, 2.0, 3.0, 4.0, 5.0, 6.0, 7.0, 8.0, 10.0, 12.0, 14.0, 16.0, or 18.0 μg/kg body weight administered weekly, every other week, or once a month. Furthermore, a therapeutically effective amount of a compound may be, for example, in the range of 100 μg/m² to 100,000 μg/m² administered every other day, once weekly, or every other week. In a desirable embodiment, the therapeutically effective amount is in the range of 1000 μg/m² to 20,000 μg/m², for example, at least 1000, 1500, 4000, or 14,000 μg/m² of the compound administered daily, every other day, twice weekly, weekly, or every other week.

All patents, patent applications, and publications mentioned in this specification are herein incorporated by reference to the same extent as if each independent patent, patent application, or publication was specifically and individually indicated to be incorporated by reference.

What is claimed is:

1. A method of diagnosing a metabolic disorder, or a propensity thereto, in a subject, said method comprising analyzing the level of sirtuin2 expression or activity in a sample isolated from said subject, wherein a decreased level of sirtuin2 expression or activity in said sample relative to the level in a control sample indicates that said subject has said metabolic disorder, or a propensity thereto.

2. The method of claim 1, wherein said analyzing comprises measuring the amount of sirtuin2 RNA or protein in said sample.

3. The method of claim 1, wherein said analyzing comprises measuring the histone deacetylase activity of sirtuin2 in said sample.

4. The method of claim 1, wherein said metabolic disorder is obesity.

5. The method of claim 1, wherein said subject is a human.

6. A method of identifying a candidate compound useful for treating a metabolic disorder in a subject, said method comprising:
(a) contacting a sirtuin2 protein, or a fragment thereof, with a compound; and
(b) measuring the activity of said sirtuin2, wherein an increase in sirtuin2 activity in the presence of said compound relative to sirtuin2 activity in the absence of said compound identifies said compound as a candidate compound for treating a metabolic disorder in a subject.

7. The method of claim 6, wherein said compound is selected from a chemical library.

8. The method of claim 6, wherein said sirtuin2 protein is human sirtuin2 protein.

9. The method of claim 6, wherein said method is performed in a cell.

10. The method of claim 6, wherein said method is performed in vitro.

11. The method of claim 6, wherein said metabolic disorder is obesity.

12. A method of identifying a candidate compound useful for treating a metabolic disorder in a subject, said method comprising:
(a) contacting a sirtuin2 protein, or a fragment thereof, with a compound; and
(b) measuring the binding of said compound to sirtuin2, wherein specific binding of said compound to said sirtuin2 protein identifies said compound as a candidate compound for treating a metabolic disorder in a subject.

13. The method of claim 12, wherein said compound is selected from a chemical library.

14. The method of claim 12, wherein said sirtuin2 protein is human sirtuin2 protein.

15. The method of claim 12, wherein said metabolic disorder is obesity.

16. A method of identifying a candidate compound useful for treating a metabolic disorder in a subject, said method comprising:
(a) contacting a cell or cell extract comprising a polynucleotide encoding sirtuin2 with a compound; and
(b) measuring the level of sirtuin2 expression in said cell or cell extract, wherein an increased level of sirtuin2 expression in the presence of said compound relative to the level in the absence of said compound identifies said compound as a candidate compound for treating a metabolic disorder in a subject.

17. The method of claim 16, wherein said candidate compound is selected from a chemical library.

18. The method of claim 16, wherein said sirtuin2 is human sirtuin2.

19. The method of claim 16, wherein said metabolic disorder is obesity.

20. A method of treating a metabolic disorder in a subject, said method comprising administering to said subject a therapeutically effective amount of a composition that increases sirtuin2 expression or activity.

21. The method of claim 20, wherein said composition comprises sirtuin2, or a fragment thereof having sirtuin2 activity.

22. The method of claim 20, wherein said composition comprises a polynucleotide encoding sirtuin2 or a fragment thereof having sirtuin2 activity.

23. The method of claim 20, wherein said composition comprises resveratrol or a derivative thereof.

24. The method of claim 20, wherein said composition comprises an antibody that specifically binds sirtuin2, or is a sirtuin2-binding fragment thereof.

25. The method of claim 20, wherein said metabolic disorder is obesity.

26. The method of claim 20, wherein said subject is a human.

27. A kit for treating a subject with a metabolic disorder, said kit comprising:
(a) a composition that increases sirtuin2 expression or activity; and
(b) instructions for administering said composition to a subject with a metabolic disorder.
